SEQUENCE LISTING (5 sequences)

SEQ ID NO: 1 - protein, 11 aa, Artificial Sequence
  Cell-penetrating Peptide
KLKLALALALA

SEQ ID NO: 2 - protein, 12 aa, Artificial Sequence
  Cell-penetrating Peptide
KLKLALALALAC

SEQ ID NO: 3 - protein, 41 aa, Homo sapiens
STFWAYQPDGDNDPTDYQKYEHTSSPSQLLAPGDYPCVIEV

SEQ ID NO: 4 - protein, 21 aa, Homo sapiens
GIVEQCCASVCSLYQLENYCN

SEQ ID NO: 5 - protein, 30 aa, Homo sapiens
FVNQHLCGSHLVEALYLVCGERGFFYTPKA

(19) United States
(12) Patent Application Publication
(10) Pub. No.: US 2010/0160209 A1
(43) Pub. Date: Jun. 24, 2010

(54) Composition and Method for Increasing Cell Permeability of a Compound

(75) Inventors: Dany Doucet, King of Prussia, PA (US); Mickey Lee Wells, Research Triangle Park, NC (US)

Correspondence Address: GlaxoSmithKline;GLOBAL PATENTS -US, UW2220, P. O. BOX 1539, KING OF PRUSSIA, PA, 19406-0939, US

(73) Assignee: SmithKline Beecham Corporation, Philadelphia, PA (US)

(21) Appl. No.: 12/300,181

(22) PCT Filed: May 10, 2007
(86) PCT No.: PCT/US07/68638

§ 371 Date: Nov. 10, 2008

Related U.S. Application Data
(60) Provisional application No. 60/799,191, filed on May 10, 2006.

(51) Int. Cl. A61K 38/28; C07K 14/62; C07K 14/585; C07K 14/575; A61K 38/29; C07K 14/635; A61K 38/22; A61K 38/23; A61P 3/10; A61P 3/00

(52) U.S. Cl. 514/3; 530/303; 530/307; 530/313; 514/12; 530/324

(57) ABSTRACT

The invention provides for a cell permeable peptide conjugated to an insulin compound for improved cell penetration of the insulin moiety. The composition may be delivered by intravenous, intramuscular, subcutaneous, intranasal, oral inhalation, intrarectal, intravaginal or intraperitoneal means for the treatment, including prophylaxis of Type I, Type II diabetes, prediabetes and/or metabolic syndrome.

### FIELD OF THE INVENTION

The present invention relates to use of cell permeable peptides and their use with small molecules and large peptides for treatment of diseases.

### BACKGROUND OF THE INVENTION

The delivery of biologically active molecules, in particular peptides to the interior of cells, has remained a problem despite various methods which have been employed. Peptides, and many small molecules do not readily cross biological membranes to enter cells. Thus, current methods which include permeabilization of the cell membrane or microinjection into the cell have been tried. Permeabilization of cells, e.g., by saponin, bacterial toxins, calcium phosphate, electroportation, etc., can only be practically useful for ex vivo methods, and these methods can cause damage to the cells. Microinjection requires highly skilled technicians, can physically damage the cells, and has only limited applications as it cannot be used to treat, for example, a mass of cells or an entire tissue, because one cannot feasibly inject large numbers of cells. Similarly, delivery of nucleic acids has also been problematic. Methods currently employed include the permeabilization method described above, as well as vector-based delivery, such as with viral vectors, and liposome-mediated delivery. Viral vectors can present additional risks to a patient, and liposome techniques have not achieved satisfactorily high levels of delivery into cells.

Signal peptide sequences, which generally share the common motif of hydrophobicity, mediate translocation of most intracellular secretory proteins across mammalian endoplasmic reticulum (ER) and prokaryotic plasma membranes through the putative protein-conducting channels. Alternative models for secretory protein transport also support a role for the signal sequence in targeting proteins to membranes.

Several types of signal sequence-mediated inside-out membrane translocation pathways have been proposed. Modeling has implied that the proteins are transported across membranes through a hydrophilic protein-conducting channel formed by a number of membrane proteins. In eukaryotes, newly synthesized proteins in the cytoplasm are targeted to the ER membrane by signal sequences that are recognized generally by the signal recognition particle (SRP) and its ER membrane receptors. This targeting step is followed by the actual transfer of protein across the ER membrane and out of the cell through the putative protein-conducting channel. In bacteria, the transport of most proteins across the cytoplasmic membrane also requires a similar protein-conducting channel. On the other hand, signal peptides can interact strongly with lipids, supporting the proposal that the transport of some secretory proteins across cellular membranes may occur directly through the lipid bilayer in the absence of any proteinaceous channels.

Using genetic engineering of proteins, Rojas, M., et al., Nature Biotechnology, Vol. 16, pgs 370-375, (1998) discloses the generation of proteins with inherent cell membrane—translocating activity, e.g. permeability. However, this paper does not address the use of a small peptide to enhance the uptake of an active agent uptake into a cell where the agent is associated with the peptide in some manner. This was partially addressed by O'Mahony et al., U.S. Pat. No. 6,780,846 which provided for a particular membrane translocating peptide (“MTLP”) complex with an active agent or particle to move across a lipid membrane.

In further developments, Lin et al., U.S. Pat. Nos. 5,807,746, 6,043,339, and 6,495,518 describe an importation component signal peptide to assist in the importation of molecules into the cell. The Lin et al. patents describe particular importation component signal peptides.

Clearly, many attempts have been made to develop effective methods for importing biologically active molecules into cells, both in vivo and in vitro, though none have proved to be entirely satisfactory. This problem affects a wide variety of therapies. The solution of this problem would greatly expand treatments of diseases for which delivery of a therapeutic molecule would be beneficial. Their still remains a long-felt need to providing a method of importing a biologically active molecules into a cell using mechanisms naturally occurring in cells and thus avoiding damaging the target cells.

### SUMMARY OF THE INVENTION

The present invention is directed to a conjugate comprising a) a cell-permeable peptide of about 11 to about 50 amino acid comprising at least one residue of SEQ ID NO: 1 and b) an insulin compound, calcitonin, calcitonin gene related peptide, parathyroid hormone, or luteinizing hormone releasing factor (LCRF). In another embodiment, the cell permeable peptide comprises at least two or more residues of SEQ ID NO: 1. In another embodiment of the invention, the residues are consecutive.

Another aspect of the invention is a pharmaceutical composition comprising a conjugate of a cell-permeable peptide of about 11 to about 50 amino acids comprising at least one residue of SEQ ID NO: 1 and b) an insulin compound, calcitonin, calcitonin gene related peptide, parathyroid hormone, or luteinizing hormone releasing factor (LCRF), admixed with a pharmaceutically acceptable carrier or diluent. In another embodiment, the cell permeable peptide portion of the conjugate comprises at least two or more residues of SEQ ID NO: 1. In another embodiment the residues are consecutive.

Another aspect of the invention is the use of the conjugate or pharmaceutical composition thereof for the treatment, or prophylaxis where appropriate, of diabetes mellitus, Type I and Type II diabetes, pre-diabetes, and metabolic syndrome where the polypeptide or protein is an insulin compound.

Another aspect of the invention is the use of a cell-permeable peptide of about 11 to about 50 amino residues comprising at least one residue of SEQ ID NO: 1 and b) an insulin compound, calcitonin, calcitonin gene related peptide, parathyroid hormone, or luteinizing hormone releasing factor (LCRF) and/or suitable protein, to enhance the uptake of said compound and/or protein into a cell.

Another aspect of the invention is a pharmaceutical composition comprising a cell-permeable peptide of about 11 to about 50 amino residues comprising at least one residue of SEQ ID NO: 1 and b) an effective amount of an insulin compound, calcitonin, calcitonin gene related peptide, parathyroid hormone, or luteinizing hormone releasing factor (LCRF) and/or suitable protein, an a pharmaceutically acceptable carrier or diluent.

### BRIEF DESCRIPTION OF THE DRAWINGS

FIG. 1 is a graphic description of the two chains of human insulin [Seq ID No. 4 and Seq ID No. 5].

FIG. 2 demonstrates an HPLC analysis of the 12 mer-CPS-CYS peptide of Seq ID No.:2 and FIG. 2a demonstrates the mass spectrometry analysis of this synthetic CPS-Cys peptide.

FIG. 3 demonstrates a mass spectrometry analysis of human insulin.

FIG. 4A demonstrates the conjugation reaction of CPS-Cys peptide to insulin via Sulfo-MBS was monitored by analytical HPLC. FIG. 4B demonstrates the conjugated product Insulin-CPS purified by HPLC which shows a retention time greater than that of unconjugated insulin. FIG. 4C demonstrates the mass spectrometry analysis of the HPLC fraction (on panel B) showed a molecular mass of 7237 Da (MH+) consistent with the calculated MW of Insulin-CPS.

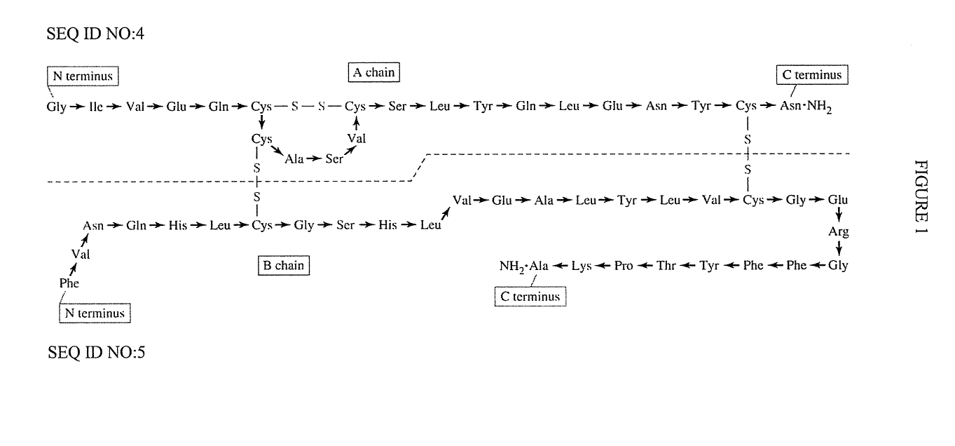

### DETAILED DESCRIPTION OF THE INVENTION

The present invention is directed to use of a novel cell-penetrating peptide sequence (CPS) Lys-Leu-Lys-Leu-Ala-Leu-Ala-Leu-Ala-Leu-Ala-COOH [Seq ID No. 1] and the novel cell penetrating peptide sequence (CPS2) 12 mer peptide K-L-K-L-A-L-A-L-A-L-A-C [Seq ID No. 2] that is operably linked to larger peptides to affect target cells, and cellular factors.

This invention provides for a method of treating insulin deficiencies or otherwise supplementing insulin in a subject in need thereof, using an insulin conjugate and/or a formulation of this invention. The methods generally include administering a therapeutically effective amount of one or more of the insulin conjugates, and/or formulations to the subject.

The present invention provides for use of an 11-mer cell-permeable peptide (CPP) as described in U.S. Ser. No. 11/270,295, filed Nov. 9, 2004, whose disclosure is incorporated herein by reference in its entirety, functionally attached to insulin or any other suitable polypeptide or protein, or small molecule as desired. As noted herein the 11 mer CPP peptide has been modified for conjunction to peptides, such as by addition of a Cys residue at the C-terminus. This yields the novel 12 mer peptide of Seq ID No. 2. Suitable polypeptide species which are covalently or associatively conjugatable in the manner of the invention, include, but are not limited to, the following species: insulin, parathyroid hormone, calcitonin, calcitonin gene regulating protein, luminal cholecystokinin releasing factor (LCRF), ACTH, glucagon, somatostain, somatotropin, somatomedin, parathyroid erythropoietin (EPO), hypothalmic releasing factors, prolactin, thyroid stimulating hormone, leuteinizing hormone releasing hormone (LHRH), growth hormone releasing hormone (GHRF), factor VIII, tissue plasminogen activator (TPA), endorphins, antibodies, hemoglobin, soluble CD-4, clotting factors, tissue plasminogen activator, enkephalins, vasopressin, non-naturally occurring opioids, superoxide dismutase, interferon, asparaginase, arginase, arginine deaminease, adenosine deaminase ribonuclease, trypsin, chemotrypsin, and papain, alkaline phosphatase, and other suitable enzymes, or hormone and proteins. Some additional hormones include but are not limited to oxytocin, estradiol, leuprolide acetate, testosterone and analogs thereof.

It is also recognized that the CPS can be attached to a small molecule for improved cell permeability. Suitable small molecules include but are not limited to anticancer agents, such as topotecan, or aromatose inhibitors, such as tamoxifen, anastrozole, letrozole, and raloxitene; protease inhibitors and other retroviral agents; and bisphosphonates, such as alendronate, ibandronate and risedronate.

The novel cell-penetrating sequence (CPS) is functionally attached to the desired peptide, and when delivered to a cell, is believed to provide for increased cell permeability of the peptide. The CPS comprises at least 11 amino acids of the sequence Lys-Leu-Lys-Leu-Ala-Leu-Ala-Leu-Ala-Leu-Ala, (SEQ ID NO: 1), or at least 12 amino acids of the sequence K-L-K-L-A-L-A-L-A-L-A-C [Seq ID No. 2]. The CPS can appear in one or more repeating sequence units in a peptide of from about 11 to about 50 or so amino acid residues. The repeating sequence of 11 amino acids (e.g. the unit) can be separated by one or more amino acids in the region between the repeating sequences of the amino acids.

Suitably, the CPS peptide contains at least one repeating sequence of the 11 amino acids. In another embodiment, the CPS peptide can include at least 2 repeating sequences of the 11 amino acids. In another embodiment, the CPS peptide can include at least 3 repeating sequences of 11 amino acids, and lastly in another embodiment, the CPS peptide can include at least 4 repeating sequence of 11 amino acids. The repeating sequence of amino acids can be separated by one or more amino acids in the region between the repeating sequences of the amino acids. The CPS peptide may also comprise additional amino acids either at the C-terminal or N-terminal end of the CPS peptide, or both, in addition to those spread throughout the CPS peptide. The addition of additional amino acids at the end of the peptide may provide for more varied coupling of the peptide with a larger polypeptide or small molecule.

It is also recognized that an amino acid residue can be substituted by another amino acid residue or its analogue.

As noted above, the CPS conjugate includes a larger polypeptide or protein, such as insulin. The insulin component may for example, be a mammalian insulin compound, such as human insulin, a native insulin, or an insulin analog.

“Native insulin compound” as specifically used herein means mammalian insulin compound (e.g., human insulin, bovine insulin compound, porcine insulin compound or whale insulin compound), provided by natural, synthetic, or genetically engineered sources. Human insulin is comprised of a twenty-one amino acid A-chain and a thirty-amino acid B-chain which are cross-linked by disulfide bonds. A properly cross-linked human insulin includes three disulfide bridges: one between A7 and B7, a second between A20 and B19, and a third between A6 and A11. Human insulin possesses three free amino groups: B1-Phenylalanine, A1-Glycine, and B29-Lysine. The free amino groups at positions A1 and B1 are α-amino groups. The free amino group at position B29 is an ε-amino group.

“Insulin analog” means a polypeptide exhibiting some, all or enhanced activity relative to a corresponding native insulin or which is converted in in vivo or in vitro into a polypeptide exhibiting one, all or enhanced activity relative to a corresponding native insulin, e.g., a polypeptide having the structure of a human insulin with one or more conservative amino acid additions, deletions and/or substitutions. Insulin analogs can be identified using known techniques, such as those described in U.S. patent Publication No. 20030049654, “Protein design automation for protein libraries,” filed 18 Mar. 2002 in the name of Dahiyat et al. Proinsulins, pre-proinsulins, insulin precursors, single chain insulin precursors of humans and non-human animals and analogs of any of the foregoing are also referred to herein as insulin analogs, as are non-mammalian insulin's. Many insulin analogs are known in the art. Unless context specifically indicates otherwise (e.g., where a specific insulin is referenced, such as “human insulin” or the like), the term “insulin” or “insulin compound” is used broadly to include native insulin's and insulin analogs.

Suitable insulin analogs are those which include a lysine, preferably a lysine within 5 amino acids of the C-terminus of the B chain, e.g. at position B26, B27, B28, B29 and/or B30. A set of suitable analogs has previously been described in the art having the sequence of an insulin compound, except that the amino acid residue at position B28 is Asp, Lys, Leu, Val, or Ala; the amino acid residue at position B29 is Lys or Pro; the amino acid residue at position B10 is His or Asp; the amino acid residue at position B1 is Phe, Asp, or deleted alone or in combination with a deletion of the residue at position B2; the amino acid residue at position B30 is Thr, Ala, or deleted; and the amino acid residue at position B9 is Ser or Asp; provided that either position B28 or B29 is Lys.

Other examples of suitable insulin analogs include Asp^{B28} human insulin, Lys^{B28} human insulin, Leu^{B28} human insulin, Val^{B28} human insulin, Ala^{B28} human insulin, Asp^{B28}Pro^{B29} human insulin, Lys^{B28}Pro^{B29} human insulin, Leu^{B28}Pro^{B29} human insulin, Val^{B28}Pro^{B29} human insulin, Ala^{B28}Pro^{B29} human insulin, as well as analogs provided using the substitution guidelines described above. Insulin compound fragments include, but are not limited to, B22-B30 human insulin, B23-B30 human insulin, B25-B30 human insulin, B26-B30 human insulin, B27-B30 human insulin, B29-B30 human insulin, B1-B2 human insulin, B1-B3 human insulin, B1-B4 human insulin, B1-B5 human insulin, the A chain of human insulin, and the B chain of human insulin.

Still other examples of suitable insulin compound analogs can be found in U.S. patent Publication No. 20030144181A1, entitled “Insoluble compositions for controlling blood glucose,” 31 Jul. 2003; U.S. patent Publication No. 20030104983A1, entitled “Stable insulin formulations,” 5 Jun. 2003; U.S. patent Publication No. 20030040601A1, entitled “Method for making insulin precursors and insulin analog precursors,” 27 Feb. 2003; U.S. patent Publication No. 20030004096A1, entitled “Zinc-free and low-zinc insulin preparations having improved stability,” 2 Jan. 2003; U.S. Pat. No. 6,551,992B1, entitled “Stable insulin formulations,” 22 Apr. 2003; U.S. Pat. No. 6,534,288B1, entitled “C peptide for improved preparation of insulin and insulin analogs,” 18 Mar. 2003; U.S. Pat. No. 6,531,448B1, entitled “Insoluble compositions for controlling blood glucose,” 11 Mar. 2003; U.S. Pat. No. RE37,971E, entitled “Selective acylation of epsilon-amino groups,” 28 Jan. 2003; U.S. patent Publication No. 20020198140A1, entitled “Pulmonary insulin crystals,” 26 Dec. 2002; U.S. Pat. No. 6,465,426B2, entitled “Insoluble insulin compositions,” 15 Oct. 2002; U.S. Pat. No. 6,444,641B1, entitled “Fatty acid-acylated insulin analogs,” 3 Sep. 2002; U.S. patent Publication No. 20020137144A1, entitled “Method for making insulin precursors and insulin precursor analogues having improved fermentation yield in yeast,” 26 Sep. 2002; U.S. patent Publication No. 20020132760A1, entitled “Stabilized insulin formulations,” 19 Sep. 2002; U.S. patent Publication No. 20020082199A1, entitled “Insoluble insulin compositions,” 27 Jun. 2002; U.S. Pat. No. 6,335,316B1, entitled “Method for administering acylated insulin,” 1 Jan. 2002; U.S. Pat. No. 6,268,335B1, entitled “Insoluble insulin compositions,” 31 Jul. 2001; U.S. patent Publication No. 20010041787A1, entitled “Method for making insulin precursors and insulin precursor analogues having improved fermentation yield in yeast,” 15 Nov. 2001; U.S. patent Publication No. 20010041786A1, entitled “Stabilized acylated insulin formulations,” 15 Nov. 2001; U.S. patent Publication No. 20010039260A1, entitled “Pulmonary insulin crystals,” 8 Nov. 2001; U.S. patent Publication No. 20010036916A1, entitled “Insoluble insulin compositions,” 1 Nov. 2001; U.S. patent Publication No. 20010007853A1, entitled “Method for administering monomeric insulin analogs,” 12 Jul. 2001; U.S. Pat. No. 6,051,551A, entitled “Method for administering acylated insulin,” 18 Apr. 2000; U.S. Pat. No. 6,034,054A, entitled “Stable insulin formulations,” 7 Mar. 2000; U.S. Pat. No. 5,970,973A, entitled “Method of delivering insulin lispro,” 26 Oct. 1999; U.S. Pat. No. 5,952,297A, entitled “Monomeric insulin analog formulations,” 14 Sep. 1999; U.S. Pat. No. 5,922,675A, entitled “Acylated Insulin Analogs,” 13 Jul. 1999; U.S. Pat. No. 5,888,477A, entitled “Use of monomeric insulin as a means for improving the bioavailability of inhaled insulin,” 30 Mar. 1999; U.S. Pat. No. 5,873,358A, entitled “Method of maintaining a diabetic patient's blood glucose level in a desired range,” 23 Feb. 1999; U.S. Pat. No. 5,747,642A, entitled “Monomeric insulin analog formulations,” 5 May 98; U.S. Pat. No. 5,693,609A, entitled “Acylated insulin compound analogs,” 2 Dec. 1997; U.S. Pat. No. 5,650,486A, entitled “Monomeric insulin analog formulations,” 22 Jul. 1997; U.S. Pat. No. 5,646,242A, entitled “Selective acylation of epsilon-amino groups,” 8 Jul. 1997; U.S. Pat. No. 5,597,893A, entitled “Preparation of stable insulin analog crystals,” 28 Jan. 1997; U.S. Pat. No. 5,547,929A, entitled “Insulin analog formulations,” 20 Aug. 1996; U.S. Pat. No. 5,504,188A, entitled “Preparation of stable zinc insulin compound analog crystals,” 2 Apr. 1996; U.S. Pat. No. 5,474,978A, entitled “Insulin analog formulations,” 12 Dec. 1995; U.S. Pat. No. 5,461,031A, entitled “Monomeric insulin analog formulations,” 24 Oct. 1995; U.S. Pat. No. 4,421,685A, entitled “Process for producing an insulin,” 20 Dec. 1983; U.S. Pat. No. 6,221,837, entitled “Insulin derivatives with increased zinc binding” 24 Apr. 2001; U.S. Pat. No. 5,177,058, entitled “Pharmaceutical formulation for the treatment of diabetes mellitus” 5 Jan. 1993 (describes pharmaceutical formulations including an insulin compound derivative modified with a base at B31 and having an isoelectric point between 5.8 and 8.5 and/or at least one of its physiologically tolerated salts in a pharmaceutically acceptable excipient, and a relatively high zinc ion content in the range from above 1 μg to about 200 μg of zinc/IU, including insulin compound-B31-Arg-OH and human insulin-B31-Arg-B32-Arg-OH). The entire disclosure of each of the foregoing patent documents is incorporated herein by reference.

The insulin component used to prepare the insulin conjugates herein can be prepared by any of a variety of recognized peptide synthesis techniques, e.g., classical (solution) methods, solid phase methods, semi-synthetic methods, and recombinant DNA methods. For example, Chance et al., U.S. patent application Ser. No. 07/388,201, EP0383472, Brange et al., EP0214826, and Belagaje et al., U.S. Pat. No. 5,304,473 discloses the preparation of various proinsulin compound and insulin compound analogs and is incorporated by reference herein. The A and B chains of the insulin compound analogs may also be prepared via a proinsulin compound-like precursor molecule or single chain insulin compound precursor molecule using recombinant DNA techniques. See Frank at al., “Peptides: Synthesis-Structure-Function,” Proc. Seventh Am. Pept. Symp., Eds. D. Rich and E. Gross (1981); Bernd Gutte, Peptides: Synthesis, Structures, and Applications, Academic Press (Oct. 19, 1995); Chan, Weng and White, Peter (Eds.), Fmoc Solid Phase Peptide Synthesis. A Practical Approach, Oxford University Press (March 2000); the entire disclosures of which are incorporated herein by reference for their teachings concerning peptide synthesis, recombinant production and manufacture.

There have been a number of efforts in the art to provide for an oral form of insulin focusing on providing an insulin conjugate. Human insulin and many closely related insulin's, such as those discussed above, used therapeutically contain three amino acid residues bearing free primary amino groups. All three primary amino groups, namely the N-termini (alpha amino groups) of the A and B chains (Gly A1 and Phe B1) and the epsilon-amino group of Lys B29, may be modified by conjugation with a conjugate such as the CPS peptide described herein. Depending on the reaction conditions, N-acylation of an unprotected insulin leads to a complex mixture of mono-, di-, and tri-conjugates (e.g., insulin mono-conjugated at GlyA1, insulin mono-conjugated at PheB1, insulin mono-conjugated at Lys B29, insulin conjugated at GlyA1 and PheB1, insulin di-conjugated at Gly A1 and Lys B29, insulin di-conjugated at PheB1 and LysB29, and insulin tri-conjugated at Gly A1, Phe B1, and Lys B29.

Various efforts have been undertaken to selectively synthesize insulin conjugates. For example, Muranishi and Kiso, in Japanese Patent Application 1-254,699, propose a five-step synthesis for preparing fatty acid insulin derivatives. The A1- and B1-amino groups of insulin are protected (or blocked) with p-methoxybenzoxy carbonyl azide (pMZ). After acylation with a fatty acid ester, the protection (blocking) groups are removed to provide insulin mono-acylated at Lys B29 with a fatty acid. As another example, U.S. Pat. No. 5,750,497 to Havelund et al. proposes treating human insulin with a Boc-reagent (e.g. di-tert-butyl dicarbonate) to form (A1, B1)-diBoc human insulin, i.e., human insulin in which the N-terminal end of both the A- and B-chains are protected by a Boc-group. After an optional purification, e.g. by HPLC, a lipophilic acyl group is introduced in the amino group of Lys B29 by allowing the product to react with a N-hydroxysuccinimide ester of the formula X—O-Succinomide wherein X is the lipophilic acyl group to be introduced. In the final step, trifluoroacetic acid is used to remove the Boc-groups and the product, N epsilon B29-X human insulin, is isolated.

Preferential synthesizing of the desired insulin conjugate as a mixture of conjugates has been undertaken by U.S. Pat. No. 5,646,242 Baker et al. in which they propose a reaction that is performed without the use of amino-protecting groups. Baker utilizes a reaction of an activated fatty ester with the ε-amino group of insulin under basic conditions in a polar solvent. The acylation of the epsilon-amino group is dependent on the basicity of the reaction. At a pH greater than 9.0, the reaction preferentially acylates the epsilon-amino group of B29-lysine over the .alpha.-amino groups. Examples 1 through 4 report reaction yields of the mono-conjugated insulin as a percentage of the initial amount of insulin between 67.1% and 75.5%. In Example 5, Baker also proposes acylation of human proinsulin with N-succinimidyl palmitate. The exact ratios of epsilon-amino acylated species to alpha-amino acylated species were not calculated. The sum of all epsilon-amino acylated species within the chromatogram accounted for 87-90% of the total area, while the sum of all related substances (which would presumably include any alpha-amino acylated species) accounted for <7% of the total area, for any given point in time. While such synthesis of an insulin—CPS conjugate is desired, it is not necessary for practicing of the invention herein.

The insulin conjugate requires the coupling of the cell permeability peptide to the insulin compound to provide the insulin conjugate. By modifying the insulin compound this will provide a conjugate with desired properties as described herein. The modified insulin will have its cell penetrating ability preferentially improved over the non-insulin conjugate. It is also an expectation that the conjugate will potentially reduce the rate of degradation of the insulin compound in vivo such that less of the insulin compound is degraded in the modified form than would be degraded in the absence of the modifying moiety in such an environment. This would permit the insulin conjugate to retain a therapeutically significant percentage of the biological activity of the parent insulin compound.

The modifying moiety of the invention, e.g. the CPS peptide may be coupled to the polypeptide or small molecule, for example, to an insulin compound, such as a human insulin, at any available point of attachment. A preferred point of attachment in the insulin example is a nucleophilic residue, e.g., A1, B1 and/or B29.

In some cases, the CPS may be coupled to the polypeptide via an amino acid or series of 2 or more amino acids coupled to the C-terminus, or a side chain of the polypeptide. For example, in one embodiment, the CPS is coupled at the —OH or —C(O)OH of Thr, and the mm-modified Thr is coupled to a polypeptide at the carboxy terminus. For example, in one embodiment, the modifying moiety is coupled at the —OH or —C(O)OH of Thr, and the modified Thr is coupled to the B29 amino acid (e.g., a B29 Lys for human insulin) of des-Thr insulin compound. In another example, the mm is coupled at the —OH or —C(O)OH of Thr of a terminal octapeptide from the insulin compound B-chain, and the mm-modified octapeptide is coupled to the B22 amino acid of des-octa insulin compound. Other variations will be apparent to one skilled in the art in light of this specification.

Factors such as the degree of conjugation with CPS, and selection of conjugation sites on the polypeptide molecule may be varied to produce a conjugate which, for example, is less susceptible to in vivo degradation, or has improved cell permeability as compared to the parent insulin moiety. For example, the insulin compound may be modified to include a CPS peptide at one, two, three, four, five, or more sites on the insulin compound structure at appropriate attachment (i.e., modifying moiety conjugation) sites suitable for facilitating the association of a modifying moiety thereon. By way of example, such suitable conjugation sites may comprise an amino acid residue, such as a lysine amino acid residue.

In some embodiments, the insulin compound conjugate will be a monoconjugate. In other embodiments, the insulin compound conjugates will be multi-conjugates, such as di-conjugates, tri-conjugates, tetra-conjugates, or penta-conjugates, and the like. The number of modifying moieties on the insulin compound is limited only by the number of conjugation sites on the insulin compound. In still other embodiments, the insulin compound conjugates will be a mixture of mono-conjugates, di-conjugates, tri-conjugates, tetra-conjugates, and/or penta-conjugates of CPS having differing numbers of repeating sequence units.

Preferred conjugation strategies are those which yield a conjugate retaining some or all of the bioactivity of the parent insulin compound. Preferred attachment sites include A1 N-terminus, B1 N-terminus, and B29 lysine side chain. The B29 monoconjugate and B1, B29 diconjugates are preferred. Another preferred point of attachment is an amino functionality on a C-peptide component or a leader peptide component of the insulin compound.

The CPS is preferably covalently coupled to the insulin compound. As noted, more than one CPS peptide may be covalently coupled to the insulin compound. Coupling may employ hydrolyzable or non-hydrolyzable bonds or mixtures of the two (i.e., different bonds at different conjugation sites). A hydrolyzable bond is an ester, carbonate or hydrolyzable carbamate bond. Use of a hydrolyzable coupling is believed to provide an insulin compound conjugate that will act as a prodrug. A prodrug approach may be desirable where the insulin compound-modifying moiety conjugate is inactive (i.e., the conjugate lacks the ability to affect the body through the insulin compound's primary mechanism of action), such as when the modifying moiety conjugation site is in a binding region of insulin compound.

In other embodiments, the insulin compound is coupled to CPS utilizing a non-hydrolyzable bond (e.g., a non-hydrolyzable carbamate, amide, or ether bond). Use of a non-hydrolyzable bond may be preferable when it is desirable to allow therapeutically significant amounts of the insulin compound conjugate to circulate in the bloodstream for an extended period of time. Bonds used to covalently couple the insulin compound to the modifying moiety in a non-hydrolyzable fashion are typically selected from the group consisting of covalent bond(s), ester moieties, carbonate moieties, carbamate moieties, amide moieties and secondary amine moieties.

CPS may be coupled to the insulin compound at various nucleophilic residues, including, but not limited to, nucleophilic hydroxyl functions and/or amino functions. Nucleophilic hydroxyl functions may be found, for example, at serine and/or tyrosine residues, and nucleophilic amino functions may be found, for example, at histidine and/or Lys residues, and/or at the one or more N-terminus of the A or B chains of the insulin compound. When the CPS peptide is coupled to the N-terminus of the natriuretic peptide, coupling preferably forms a secondary amine. CPS may also be coupled to the insulin compound at a free —SH group, e.g., by forming a thioester, thioether or sulfonate bond. CPS may be coupled to the insulin compound via one or more amino groups. Examples in human insulin include the amino groups at A1, B1 and B29. In one embodiment, a single CPS moiety is coupled to a single amino group on the insulin compound. In another embodiment, two CPS moieties are each connected to a different amino group on the insulin compound. Where there are two CPS moieties coupled to two amino groups, a preferred arrangement is coupling of at B1 and B29.

Another embodiment of the present invention is the coupling of one or more CPS moieties to an LCRF peptide. The coupling of the CPS moieties should preferentially not interfere with receptor binding of the LCRF molecule. WO 01/41812 discusses the LCRF conjugate with a peglyated components and indicates that residues 11 to 25 of the LCRF moiety are crucial for interaction of the molecule at the receptor and cleavage of residues 19 and 20 destroy the binding activity of the molecule. It is suggested therein that the K19 residue be protected with a hydrolyzable linker to protect it from trypsin proteolysis.

LCRF contains 2 reactive amino acid groups to use for linking the CPS peptide, the amino terminus and a lysine side chain. The N-terminus attachment can be by a non-hydrolyzable linker if desired. The second side is the epsilon amino group of K19. The amino acid sequence of LCRF is

STFWAYQPDGDNDPTDYQKYEHTSSPSQLLAPGDYPCVIEV identified as SEQ ID No. 3 herein.

Non-limiting examples of additional large protein/polypeptide that may be useful in the present invention include the following:

Adrenocorticotropic hormone (ACTH) peptides including, but not limited to, ACTH, human; ACTH 1-10; ACTH 1-13, human; ACTH 1-16, human; ACTH 1-17; ACTH 1-24, human; ACTH 4-10; ACTH 4-11; ACTH 6-24; ACTH 7-38, human; ACTH 18-39, human; ACTH, rat; ACTH 12-39, rat; beta-cell tropin (ACTH 22-39); biotinyl-ACTH 1-24, human; biotinyl-ACTH 7-38, human; corticostatin, human; corticostatin, rabbit; [Met(02)⁴, DLys⁸, Phe⁹] ACTH 4-9, human; [Met(0)⁴,DLys⁸, Phe⁹] ACTH 4-9, human; N-acetyl, ACTH 1-17, human; and ebiratide.

Adrenomedullin peptides including, but not limited to, adrenomedullin, adrenomedullin 1-52, human; adrenomedullin 1-12, human; adrenomedullin 13-52, human; adrenomedullin 22-52, human; pro-adrenomedullin 45-92, human; pro-adrenomedullin 153-185, human; adrenomedullin 1-52, porcine; pro-adrenomedullin (N-20), porcine; adrenomedullin 1-50, rat; adrenomedullin 11-50, rat; and proAM-N20 (proadrenomedullin N-terminal 20 peptide), rat.

Allatostatin peptides including, but not limited to, allatostatin I; allatostatin II; allatostatin III; and allatostatin IV.

Amylin peptides including, but not limited to, acetyl-amylin 8-37, human; acetylated amylin 8-37, rat; AC187 amylin antagonist; AC253 amylin antagonist; AC625 amylin antagonist; amylin 8-37, human; amylin (IAPP), cat; amylin (insulinoma or islet amyloid polypeptide (IAPP)); amylin amide, human; amylin 1-13 (diabetes-associated peptide 1-13), human; amylin 20-29 (IAPP 20-29), human; AC625 amylin antagonist; amylin 8-37, human; amylin (IAPP), cat; amylin, rat; amylin 8-37, rat; biotinyl-amylin, rat; and biotinyl-amylin amide, human.

Amyloid beta-protein fragment peptides including, but not limited to, Alzheimer's disease beta-protein 12-28 (SP17); amyloid beta-protein 25-35; amyloid beta/A4-protein precursor 328-332; amyloid beta/A4 protein precursor (APP) 319-335; amyloid beta-protein 143; amyloid beta-protein 1-42; amyloid beta-protein 1-40; amyloid beta-protein 10-20; amyloid beta-protein 22-35; Alzheimer's disease beta-protein (SP28); beta-amyloid peptide 1-42, rat; beta-amyloid peptide 1-40, rat; beta-amyloid 1-11; beta-amyloid 31-35; beta-amyloid 32-35; beta-amyloid 35-25; beta-amyloid/A4 protein precursor 96-110; beta-amyloid precursor protein 657-676; beta-amyloid 1-38; [Gln¹¹]-Alzheimer's Alzheimer's disease beta-protein; [Gln¹¹]-beta-amyloid 1-40; [Gln²²]-beta-amyloid 6-40; non-A beta component of Alzheimer's disease amyloid (NAC); P3, (A beta 1740) Alzheimer's disease amyloid .beta.-peptide; and SAP (serum amyloid P component) 194-204.

Angiotensin peptides including, but not limited to, A-779; Ala-Pro-Gly-angiotensin II; [Ile³,Val⁵]-angiotensin II; angiotensin III antipeptide; angiogenin fragment 108-122; angiogenin fragment 108-123; angiotensin I converting enzyme inhibitor; angiotensin I, human; angiotensin I converting enzyme substrate; angiotensin I 1-7, human; angiopeptin; angiotensin II, human; angiotensin II antipeptide; angiotensin II 1-4, human; angiotensin II 3-8, human; angiotensin II 4-8, human; angiotensin II 5-8, human; angiotensin III ([Des-Asp¹]-angiotensin II), human; angiotensin III inhibitor ([Ile⁷]-angiotensin III); angiotensin-converting enzyme inhibitor (Neothunnus macropterus); [Asn¹, Val⁵]-angiotensin I, goosefish; [Asn¹, Val⁵, Gly⁹]-angiotensin I, salmon; [Asn¹, Val⁵, Gly⁹]-angiotensin I, eel; [Asn¹, Val⁵]-angiotensin I 1-7, eel, goosefish, salmon; [Asn¹, Val⁵]-angiotensin II; biotinyl-angiotensin I, human; biotinyl-angiotensin II, human; biotinyl-Ala-Ala-Ala-angiotensin II; [Des-Asp¹]-angiotensin I, human; [p-aminophenylalanine⁶]-angiotensin II; renin substrate (angiotensinogen 1-13), human; preangiotensinogen 1-14 (renin substrate tetradecapeptide), human; renin substrate tetradecapeptide (angiotensinogen 1-14), porcine; [Sar¹]-angiotensin II, [Sar¹]-angiotensin II 1-7 amide; [Sar¹, Ala⁸]-angiotensin II; [Sar¹, Ile⁸]-angiotensin II; [Sar¹, Thr⁸]-angiotensin II; [Sar¹, Tyr(Me)⁴]-angiotensin II (Sarmesin); [Sar¹, Val⁵, Ala⁸]-angiotensin II; [Sar¹, Ile⁷]-angiotensin III; synthetic tetradecapeptide renin substrate (No. 2); [Val⁴]-angiotensin III; [Val⁵]-angiotensin II; [Val⁵]-angiotensin I, human; [Val⁵]-angiotensin I; [Val⁵Asn⁹]-angiotensin I, bullfrog; and [Val⁵, Ser⁹]-angiotensin I, fowl.

Antibiotic peptides including, but not limited to, Ac-SQNY; bactenecin, bovine; CAP 37 (20-44); carbormethoxycarbonyl-DPro-DPhe-OBz1; CD36 peptide P 139-155; CD36 peptide P 93-110; cecropin A-melittin hybrid peptide [CA(1-7)M(2-9)NH2]; cecropin B, free acid; CYS(Bzl)84 CD fragment 81-92; defensin (human) HNP-2; dermaseptin; immunostimulating peptide, human; lactoferricin, bovine (BLFC); and magainin spacer.

Antigenic polypeptides, which can elicit an enhanced immune response, enhance an immune response and or cause an immunizingly effective response to diseases and/or disease causing agents including, but not limited to, adenoviruses; anthrax; Bordetella pertussus; botulism; bovine rhinotracheitis; Branhamella catarrhalis; canine hepatitis; canine distemper; Chlamydiae; cholera; coccidiomycosis; cowpox; cytomegalovirus; Dengue fever; dengue toxoplasmosis; diphtheria; encephalitis; enterotoxigenic E. coli; Epstein Barr virus; equine encephalitis; equine infectious anemia; equine influenza; equine pneumonia; equine rhinovirus; Escherichia coli; feline leukemia; flavivirus; globulin; haemophilus influenza type b; Haemophilus influenzae; Haemophilus pertussis; Helicobacter pylori; hemophilus; hepatitis; hepatitis A; hepatitis B; Hepatitis C; herpes viruses; HIV; HIV-1 viruses; HIV-2 viruses; HTLV; influenza; Japanese encephalitis; Klebsiellae species; Legionella pneumophila; leishmania; leprosy; lyme disease; malaria immunogen; measles; meningitis; meningococcal; Meningococcal polysaccharide group A; Meningococcal polysaccharide group C; mumps; mumps virus; mycobacteria; Mycobacterium tuberculosis; Neisseria; Neisseria gonorrhea; Neisseria meningitidis; ovine blue tongue; ovine encephalitis; papilloma; parainfluenza; paramyxoviruses; Pertussis; plague; pneumococcus; Pneumocystis carinii; pneumonia; poliovirus; proteus species; Pseudomonas aeruginosa; rabies; respiratory syncytial virus; rotavirus; rubella; salmonellae; schistosomiasis; shigellae; simian immunodeficiency virus; smallpox; Staphylococcus aureus; Staphylococcus species; Streptococcus pneumoniae; Streptococcus pyogenes; Streptococcus species; swine influenza; tetanus; Treponema pallidum; typhoid; vaccinia; varicella-zoster virus; and vibrio cholerae.

Anti-microbial peptides including, but not limited to, buforin I; buforin II; cecropin A; cecropin B; cecropin P1, porcine; gaegurin 2 (Rana rugosa); gaegurin 5 (Rana rugosa); indolicidin; protegrin-(PG)-I; magainin 1; and magainin 2; and T-22 [Tyr^{5,12}, Lys⁷]-poly-phemusin II peptide.

Apoptosis related peptides including, but not limited to, Alzheimer's disease beta-protein (SP28); calpain inhibitor peptide; capsase-1 inhibitor V; capsase-3, substrate IV; caspase-1, inhibitor 1, cell-permeable; caspase-1 inhibitor VI; caspase-3 substrate III, fluorogenic; caspase-1 substrate V, fluorogenic; caspase-3 inhibitor I, cell-permeable; caspase-6 ICE inhibitor III; [Des-Ac, biotin]-ICE inhibitor III; IL-1 B converting enzyme (ICE) inhibitor II; IL-1 B converting enzyme (ICE) substrate IV; MDL 28170; and MG-132.

Atrial natriuretic peptides including, but not limited to, alpha-ANP (alpha-chANP), chicken; anantin; ANP 1-11, rat; ANP 8-30, frog; ANP 11-30, frog; ANP-21 (fANP-21), frog; ANP-24 (fANP-24), frog; ANP-30, frog; ANP fragment 5-28, human, canine; ANP-7-23, human; ANP fragment 7-28, human, canine; alpha-atrial natriuretic polypeptide 1-28, human, canine; A71915, rat; atrial natriuretic factor 8-33, rat; atrial natriuretic polypeptide 3-28, human; atrial natriuretic polypeptide 4-28, human, canine; atrial natriuretic polypeptide 5-27; human; atrial natriuretic aeptide (ANP), eel; atriopeptin I, rat, rabbit, mouse; atriopeptin II, rat, rabbit, mouse; atriopeptin III, rat, rabbit, mouse; atrial natriuretic factor (rANF), rat, auriculin A (rat ANF 126-149); auriculin B (rat ANF 126-150); beta-ANP (1-28, dimer, antiparallel); beta-rANF 17-48; biotinyl-alpha-ANP 1-28, human, canine; biotinyl-atrial natriuretic factor (biotinyl-rANF), rat; cardiodilatin 1-16, human; C-ANF 4-23, rat; Des-[Cys¹⁰⁵, Cys¹²¹]-atrial natriuretic factor 104-126, rat; [Met(O)¹²] ANP 1-28, human; [Mpr⁷,DAla⁹]ANP 7-28, amide, rat; prepro-ANF 104-116, human; prepro-ANF 26-55 (proANF 1-30), human; prepro-ANF 56-92 (proANF 31-67), human; prepro-ANF 104-123, human; [Tyr⁰]-atriopeptin I, rat, rabbit, mouse; [Tyr⁰]-atriopeptin II, rat, rabbit, mouse; [Tyr⁰]-prepro ANF 104-123, human; urodilatin (CDD/ANP 95-126); ventricular natriuretic peptide (VNP), eel; and ventricular natriuretic peptide (VNP), rainbow trout.

Bag cell peptides including, but not limited to, alpha bag cell peptide; alpha-bag cell peptide 1-9; alpha-bag cell peptide 1-8; alpha-bag cell peptide 1-7; beta-bag cell factor; and gamma-bag cell factor.

Bombesin peptides including, but not limited to, alpha-s1 casein 101-123 (bovine milk); biotinyl-bombesin; bombesin 8-14; bombesin; [Leu¹³-psi (CH2NH)Leu⁴]-bombesin; [D-Phe⁶, Des-Met¹⁴]-bombesin 6-14 ethylamide; [DPhe¹²] bombesin; [DPhe¹²,Leu¹⁴]-bombesin; [Tyr⁴]-bombesin; and [Tyr⁴, DPhe¹²]-bombesin.

Bone GLA peptides (BGP) including, but not limited to, bone GLA protein; bone GLA protein 45-49; [Glu17,Gla^{21,24}]-osteocalcin 1-49, human; myclopeptide-2 (MP-2); osteocalcin 1-49 human; osteocalcin 37-49, human; and [Tyr¹⁸, Phe^{42,46}] bone GLA protein 38-49, human.

Bradykinin peptides including, but not limited to, [Ala^{2,6}, des-Pro³]-bradykinin; bradykinin; bradykinin (Bowfin. Gar); bradykinin potentiating peptide; bradykinin 1-3; bradykinin 1-5; bradykinin 1-6; bradykinin 1-7; bradykinin 2-7; bradykinin 2-9; [DPhe⁷] bradykinin; [Des-Arg⁹]-bradykinin; [Des-Arg¹⁰]-Lys-bradykinin ([Des-Arg¹⁰]-kallidin); [D-N-Me-Phe⁷]-bradykinin; [Des-Arg9, Leu8]-bradykinin; Lys-bradykinin (kallidin); Lys-[Des-Arg⁹,Leu⁸]-bradykinin ([Des-Arg¹⁰,Leu⁹]-kallidin); [Lys¹⁰-Hyp³]-bradykinin; ovokinin; [Lys⁰, Ala³-bradykinin; Met-Lys-bradykinin; peptide K12 bradykinin potentiating peptide; [(pCl)Phe^{5,8}]-bradykinin; T-kinin (Ile-Ser-bradykinin); [Thi^{5,8}, D-Phe⁷]-bradykinin; [Tyr⁰-bradykinin; [Tyr⁵]-bradykinin; [Tyr⁸]-bradykinin; and kallikrein.

Brain natriuretic peptides (BNP) including, but not limited to, BNP 32, canine; BNP-like Peptide, eel; BNP-32, human; BNP-45, mouse; BNP-26, porcine; BNP-32, porcine; biotinyl-BNP-32, porcine; BNP-32, rat; biotinyl-BNP-32, rat; BNP-45 (BNP 51-95, 5K cardiac natriuretic peptide), rat; and [Tyr⁰]-BNP 1-32, human.

C-peptides including, but not limited to, C-peptide; and [Tyr⁰]-C-peptide, human.

C-type natriuretic peptides (CNP) including, but not limited to, C-type natriuretic peptide, chicken; C-type natriuretic peptide-22 (CNP-22), porcine, rat, human; C-type natriuretic peptide-53 (CNP-53), human; C-type natriuretic peptide-53 (CNP-53), porcine, rat; C-type natriuretic peptide-53 (porcine, rat) 1-29 (CNP-53 1-29); prepro-CNP 1-27, rat; prepro-CNP 30-50, porcine, rat; vasonatrin peptide (VNP); and [Tyr⁰]-C-type natriuretic peptide-22 ([Tyr⁰]-CNP-22).

CART peptides including, but not limited to, CART, human; CART 55-102, human; CART, rat; and CART 55-102, rat.

Calcitonin peptides including but not limited to, biotinyl-calcitonin, human; biotinyl-calcitonin, rat; biotinyl-calcitonin, salmon; calcitonin, chicken; calcitonin, eel; calcitonin, human; calcitonin, procine; calcitonin, rat; calcitonin, salmon; calcitonin 1-7, human; calcitonin 8-32, salmon; katacalin (PDN-21)(C-procalcitonin); and N-proCT (amino terminal procalcitonin cleavage peptide), human.

Calcitonin gene related peptides (CGRP) including, but not limited to, acetyl-alpha-CGRP 19-37, human; alpha-CGRP 19-37, human; alpha-CGRP 23-37, human; biotinyl-CGRP, human; biotinyl-CGRP II, human; biotinyl-CGRP, rat; beta-CGRP, rat; biotinyl-beta-CGRP, rat; CGRP, rat; CGRP, human; calcitonin C-terminal adjacent peptide; CGRP 1-19, human; CGRP 20-37, human; CGRP 8-37, human; CGRP II, human; CGRP, rat; CGRP 8-37, rat; CGRP 29-37, rat; CGRP 30-37, rat; CGRP 31-37, rat; CGRP 32-37, rat; CGRP 33-37, rat; CGRP 31-37, rat; ([Cys(Acm)^{2,7}]-CGRP; elcatonin; [Tyr⁰]-CGRP, human; [Tyr⁰]-CGRP II, human; [Tyr⁰]-CGRP 28-37, rat; [Tyr⁰]-CGRP, rat; and [Tyr²²]-CGRP 22-37, rat.

Casomorphin peptides including, but not limited to, beta-casomorphin, human; beta-casomorphin 1-3; beta-casomorphin 1-3, amide; beta-casomorphin, bovine; beta-casomorphin 1-4, bovine; beta-casomorphin 1-5, bovine; beta-casomorphin 1-5, amide, bovine; beta-casomorphin 1-6, bovine; [DAla²]-beta-casomorphin 1-3,-amide, bovine; [DAla²,Hyp⁴, Tyr⁵]-beta-casomorphin 1-5 amide; [DAla²,DPro⁴,Tyr⁵]-beta-casomorphin 1-5, amide; [DAla², Tyr⁵]-beta-casomorphin 1-5, amide, bovine; [DAla^{2,4}, Tyr⁵]-beta-casomorphin 1-5, amide, bovine; [DAla², (pCl)Phe³]-beta-casomorphin, amide, bovine; [DAla²]-beta-casomorphin 1-4, amide, bovine; [DAla²]-beta-casomorphin 1-5, bovine; [DAla²]-beta-casomorphin 1-5, amide, bovine; [DAla²,Met⁵]-beta-casomorphin 1-5, bovine; [DPro²]-beta-casomorphin 1-5, amide, bovine; [DAla²]-beta-casomorphin 1-6, bovine; [DPro²]-beta-casomorphin 1-4, amide; [Des-Tyr¹]-beta-casomorphin, bovine; [DAla²′⁴, Tyr⁵]-beta-casomorphin 1-5, amide, bovine; [DAla², (pCl)Phe³]-beta-casomorphin, amide, bovine; [DAla²]-beta-casomorphin 1-4, amide, bovine; [DAla²]-beta-casomorphin 1-5, bovine; [DAla²]-beta-casomorphin 1-5, amide, bovine; [DAla²,Met⁵]-beta-casomorphin 1-5, bovine; [DPro²]-beta-casomorphin 1-5, amide, bovine; [DAla²]-beta-casomorphin 1-6, bovine; [DPro²]-beta-1-4, amide, [Des-Tyr¹]-beta-casomorphine, bovine, and [Val³]-beta-casomorphin 1-4, amide, bovine.

Chemotactic peptides including, but not limited to, defensin 1 (human) HNP-1 (human neutrophil peptide-1); and N-formyl-Met-Leu-Phe.

Cholecystokinin (CCK) peptides including, but not limited to, caerulein; cholecystokinin; cholecystokinin-pancreozymin; CCK-33, human; cholecystokinin octapeptide 1-4 (non-sulfated) (CCK 26-29, unsulfated); cholecystokinin octapeptide (CCK 26-33); cholecystokinin octapeptide (non-sulfated) (CCK 26-33, unsulfated); cholecystokinin heptapeptide (CCK 27-33); cholecystokinin tetrapeptide (CCK 30-33); CCK-33, porcine; CR 1 409, cholecystokinin antagonist; CCK flanking peptide (unsulfated); N-acetyl cholecystokinin, CCK 26-30, sulfated; N-acetyl cholecystokinin, CCK 26-31, sulfated; N-acetyl cholecystokinin, CCK 26-31, non-sulfated; prepro CCK fragment V-9-M; and proglumide.

Colony-stimulating factor peptides including, but not limited to, colony-stimulating factor (CSF); GMCSF; MCSF; and G-CSF.

Corticortropin releasing factor (CRF) peptides including, but not limited to, astressin; alpha-helical CRF 12-41; biotinyl-CRF, ovine; biotinyl-CRF, human, rat; CRF, bovine; CRF, human, rat; CRF, ovine; CRF, porcine; [Cys²¹]-CRF, human, rat; CRF antagonist (alpha-helical CRF 9-41); CRF 6-33, human, rat; [DPro⁵]-CRF, human, rat; [D-Phe², Nle^{21,38}]-CRF 12-41, human, rat; eosinophilotactic peptide; [Met(0)²¹]-CRF, ovine; [Nle²¹,Tyr³²]-CRF, ovine; prepro CRF 125-151, human; sauvagine, frog; [Tyr⁰]-CRF, human, rat; [Tyr⁰]-CRF, ovine; [Tyr⁰]-CRF34-41, ovine; [Tyr⁰]-urocortin; urocortin amide, human; urocortin, rat; urotensin I (Catostomus commersoni); urotensin II; and urotensin II (Rana ridibunda).

Cortistatin peptides including, but not limited to, cortistatin 29; cortistatin 29 (1-13); [Tyr⁰]-cortistatin 29; pro-cortistatin 28-47; and pro-cortistatin 51-81.

Cytokine peptides including, but not limited to, tumor necrosis factor; and tumor necrosis factor-.beta. (TNF-.beta.).

Dermorphin peptides including, but not limited to, dermorphin and dermorphin analog 1-4.

Dynorphin peptides including, but not limited to, big dynorphin (prodynorphin 209-240), porcine; biotinyl-dynorphin A (biotinyl-prodynorphin 209-225); [DAla², DArg⁶]-dynorphin A 1-13, porcine; [DAla²]-dynorphin A, porcine; [DAla²-dynorphin A amide, porcine; [DAla²]-dynorphin A 1-13, amide, porcine; [DAla²]-dynorphin A 1-9, porcine; [DArg⁶]-dynorphin A 1-13, porcine; [DArg⁸]-dynorphin A 1-13, porcine; [Des-Tyr¹]-dynorphi-n A 1-8; [D-Pro¹⁰]-dynorphin A 1-11, porcine; dynorphin A amide, porcine; dynorphin A 1-6, porcine; dynorphin A 1-7, porcine; dynorphin A 1-8, porcine; dynorphin A 1-9, porcine; dynorphin A 1-10, porcine; dynorphin A 1-10 amide, porcine; dynorphin A 1-11, porcine; dynorphin A 1-12, porcine; dynorphin A 1-13, porcine; dynorphin A 1-13 amide, porcine; DAKLI (dynorphin A-analogue kappa ligand); DAKLI-biotin ([Arg^{11,13}]-dynorphin A (1-13)-Gly-NH(CH₂)₅NH-biotin); dynorphin A 2-17, porcine; dynorphin 2-17, amide, porcine; dynorphin A 2-12, porcine; dynorphin A 3-17, amide, porcine; dynorphin A 3-8, porcine; dynorphin A 3-13, porcine; dynorphin A 3-17, porcine; dynorphin A 7-17, porcine; dynorphin A 8-17, porcine; dynorphin A 6-17, porcine; dynorphin A 13-17, porcine; dynorphin A (prodynorphin 209-225), porcine; dynorphin B 1-9; [Me Tyr¹, MeArg⁷, D-Leu⁸]-dynorphin 1-8 ethyl amide; [(nMe)Tyr¹] dynorphin A 1-13, amide, porcine; [Phe⁷]-dynorphin A 1-7, porcine; [Phe⁷]-dynorphin A 1-7, amide, porcine; and prodynorphin 228-256-(dynorphin B 29) (leumorphin), porcine.

Endorphin peptides including, but not limited to, alpha-neo-endorphin, porcine; beta-neo-endorphin; Ac-beta-endorphin, camel, bovine, ovine; Ac-beta-endorphin 1-27, camel, bovine, ovine; Ac-beta-endorphin, human; Ac-beta-endorphin 1-26, human; Ac-beta-endorphin 1-27, human; Ac-gamma-endorphin (Ac-beta-lipotropin 61-77); acetyl-alpha-endorphin; alpha-endorphin (beta-lipotropin 61-76); alpha-neo-endorphin analog; alpha-neo-endorphin 1-7; [Arg⁸]-alpha-neo-endorphin 1-8; beta-endorphin (beta-lipotropin 61-91), camel, bovine, ovine; beta-endorphin 1-27, camel, bovine, ovine; beta-endorphin, equine; beta-endorphin (beta-lipotropin 61-91), human; beta-endorphin (1-5)+(16-31), human; beta-endorphin 1-26, human; beta-endorphin 1-27, human; beta-endorphin 6-31, human; beta-endorphin 18-31, human; beta-endorphin, porcine; beta-endorphin, rat; beta-lipotropin 1-10, porcine; beta-lipotropin 60-65; beta-lipotropin 61-64; beta-lipotropin 61-69; beta-lipotropin 88-91; biotinyl-beta-endorphin (biotinyl-beta-lipotropin 61-91); biocytin-beta-endorphin, human; gamma-endorphin (beta-lipotropin 61-77); [DAla²]-alpha-neo-endorphin 1-2, amide; [DAla²]-beta-lipotropin 61-69; [DAla²]-gamma-endorphin; [Des-Tyr¹]-beta-endorphin, human; [Des-Tyr¹]-gamma-endorphin (beta-lipotropin 62-77); [Leu⁵]-beta-endorphin, camel, bovine, ovine; [Met⁵, Lys⁶]-alpha-neo-endorphin 1-6; [Met⁵, Lys6,7]-alpha-neo-endorphin 1-7; and [Met⁵, Lys⁶, Arg⁷]-alpha-neo-endorphin 1-7.

Endothelin peptides including, but not limited to, endothelin-1 (ET-1); endothelin-1[Biotin-Lys⁹]; endothelin-1 (1-15), human; endothelin-1 (1-15), amide, human; Ac-endothelin-1 (16-21), human; Ac-[DTrp¹⁶]-endothelin-1 (16-21), human; [Ala^{3,11}]-endothelin-1-; [Dpr1, Asp¹⁵]-endothelin-1; [Ala²]-endothelin-3, human; [Ala¹⁸]-endothelin-1, human; [Asn¹⁸-endothelin-1, human; [Res-701-1]-endothelin B receptor antagonist; Suc-[Glu⁹, Ala^{11,15}]-endothelin-1 (8-21), endothelin-C-terminal hexapeptide; [D-Val²²]-big endothelin-1 (16-38), human; endothelin-2 (ET-2), human, canine; endothelin-3 (ET-3), human, rat, porcine, rabbit; biotinyl-endothelin-3 (biotinyl-ET-3); prepro-endothelin-1 (94-109), porcine, endothelium-dependent relaxation antagonist; sarafotoxin S6a (atractaspis engaddensis); sarafotoxin S6b (atractaspis engaddensis); sarafotoxin S6c (atractaspis engaddensis); [Lys⁴]-sarafotoxin S6c; sarafotoxin S6d; big endothelin-1, human; biotinyl-big endothelin-1, human; big endothelin-1 (1-39), porcine; big endothelin-3 (22-41), amide, human; big endothelin-1 (22-39), rat; big endothelin-1 (1-39), bovine; big endothelin-1 (22-39), bovine; big endothelin-1 (19-38), human; big endothelin-1 (22-38), human; big endothelin-2, human; big endothelin-2 (22-37), human; big endothelin-3, human; big endothelin-1, porcine; big endothelin-1 (22-39) (prepro-endothelin-1 (74-91)); big endothelin-1, rat; big endothelin-2 (1-38), human; big endothelin-2 (22-38), human; big endothelin-3, rat; biotinyl-big endothelin-1, human; and [Tyr¹²³]-prepro-endothelin (110-130), amide, human.

Enkephalin peptides including, but not limited to, adrenorphin, free acid; amidorphin (proenkephalin A (104-129)-NH2), bovine; BAM-12P (bovine adrenal medulla dodecapeptide); BAM-22P (bovine adrenal medulla docosapeptide); benzoyl-Phe-Ala-Arg; enkephalin; [DAla², D-Leu⁵]-enkephalin; [D-Ala², D-Met⁵]enkephalin; [DAla²]-Leu-enkephalin, amide; [DAla²,Leu⁵,Arg6]-enke-phalin; [Des-Tyr¹,DPen^{2,5}]-enkephalin; [Des-Tyr¹,DPen²,Pen⁵]-enkephalin; [Des-Tyr¹]-Leu-enkephalin; [D-Pen^{2,5}]-enkephalin; [DPen², Pen⁵]-enkephalin; enkephalinase substrate; [D-Pen²; pCI-Phe⁴, D-Pen⁵]-enkephalin; Leu-enkephalin; Leu-enkephalin, amide; biotinyl-Leu-enkephalin; [DAla²]-Leu-enkephalin; [D-Ser²]-Leu-enkephalin-Thr (delta-receptor peptide) (DSLET); [D-Thr¹]-Leu-enkephalin-Thr (DTLET); [Lys⁶]-Leu-enkephalin; [Met⁵,Arg⁶]-enkephalin; [Met⁵, Arg⁶]-enkephalin-Arg; [Met⁵,Arg⁶,Phe⁷]-enkephalin, amide; Met-enkephalin; biotinyl-Met-enkephalin; [DAla²]-Met-enkephalin; [DAla²]-Met-enkephalin, amide; Met-enkephalin-Arg-Phe; Met-enkephalin, amide; [DAla²]-Met-enkephalin, amide; [DMet²,Pro⁵]-enkephalin, amide; [DTrp²]-Met-enkephalin, amide, metorphinamide (adrenorphin); peptide B, bovine; 3200-Dalton adrenal peptide E, bovine; peptide F, bovine; preproenkephalin B 186-204, human; spinorphin, bovine; and thiorphan (D,L,3-mercapto-2-benzylpropan-oyl-glycine).

Fibronectin peptides including, but not limited to platelet factor-4 (58-70), human; echistatin (Echis carinatus); E, P, L selectin conserved region; fibronectin analog; fibronectin-binding protein; fibrinopeptide A, human; [Tyr⁰]-fibrinopeptide A, human; fibrinopeptide B, human; [Glu¹]-fibrinopeptide B, human; [Tyr¹⁵]-fibrinopeptide B, human; fibrinogen beta-chain fragment of 24-42; fibrinogen binding inhibitor peptide; fibronectin related peptide (collagen binding fragment); fibrinolysis inhibiting factor; FN-CH-1 (fibronectin heparin-binding fragment); FN-C/H-V (fibronectin heparin-binding fragment); heparin-binding peptide; laminin penta peptide, amide; Leu-Asp-Val-NH2 (LDV-NH2), human, bovine, rat, chicken; necrofibrin, human; necrofibrin, rat; and platelet membrane glycoprotein IIB peptide 296-306.

Galanin peptides including, but not limited to, galanin, human; galanin 1-19, human; preprogalanin 1-30, human; preprogalanin 65-88, human; preprogalanin 89-123, human; galanin, porcine; galanin 1-16, porcine, rat; galanin, rat; biotinyl-galanin, rat; preprogalanin 28-67, rat; galanin 1-13-bradykinin 2-9, amide; M40, galanin 1-13-Pro-Pro-(Ala-Leu)-2-Ala-amide; C7, galanin 1-13-spantide-amide; GMAP 1-41, amide; GMAP 16-41, amide; GMAP 25-41, amide; galantide; and entero-kassinin.

Gastrin peptides including, but not limited to, gastrin, chicken; gastric inhibitory peptide (GIP), human; gastrin I, human; biotinyl-gastrin I, human; big gastrin-1, human; gastrin releasing peptide, human; gastrin releasing peptide 1-16, human; gastric inhibitory polypeptide (GIP), porcine; gastrin releasing peptide, porcine; biotinyl-gastrin releasing peptide, porcine; gastrin releasing peptide 14-27, porcine, human; little gastrin, rat; pentagastrin; gastric inhibitory peptide 1-30, porcine; gastric inhibitory peptide 1-30, amide, porcine; [Tyr⁰]-gastric inhibitory peptide 23-42, human; and gastric inhibitory peptide, rat.

Glucagon peptides including, but not limited to, [Des-His¹,Glu⁹]-glucagon, extendin-4, glucagon, human; biotinyl-glucagon, human; glucagon 19-29, human; glucagon 22-29, human; Des-His¹-[Glu⁹]-glucagon, amide; glucagon-like peptide 1, amide (preproglucagon 72-107, amide); glucagon-like peptide 1 (preproglucagon 72-108), human; glucagon-like peptide 1 (7-36) (preproglucagon 78-107, amide); glucagon-like peptide II, rat; biotinyl-glucagon-like peptide-1 (7-36) (biotinyl-preproglucagon 78-107, amide); glucagon-like peptide 2 (preproglucagon 126-159), human; oxyntomodulin/glucagon 37; and valosin (peptide VQY), porcine.

Gn-RH associated peptides (GAP) including, but not limited to, Gn-RH associated peptide 25-53, human; Gn-RH associated peptide 1-24, human; Gn-RH associated peptide 1-13, human; Gn-RH associated peptide 1-13, rat; gonadotropin releasing peptide, follicular, human; [Tyr⁰]-GAP ([Tyr⁰]-Gn-RH Precursor Peptide 14-69), human; and proopiomelanocortin (POMC) precursor 27-52, porcine.

Growth factor peptides including, but not limited to, cell growth factors; epidermal growth factors; tumor growth factor; alpha-TGF; beta-TF; alpha-TGF 34-43, rat; EGF, human; acidic fibroblast growth factor; basic fibroblast growth factor; basic fibroblast growth factor 13-18; basic fibroblast growth factor 120-125; brain derived acidic fibroblast growth factor 1-11; brain derived basic fibroblast growth factor 1-24; brain derived acidic fibroblast growth factor 102-111; [Cys(Acm^{20,31})]-epidermal growth factor 20-31; epidermal growth factor receptor peptide 985-996; insulin-like growth factor (IGF)-I, chicken; IGF-I, rat; IGF-I, human; Des (1-3) IGF-I, human; R3 IGF-I, human; R3 IGF-I, human; long R3 IGF-I, human; adjuvant peptide analog; anorexigenic peptide; Des (1-6) IGF-II, human; R6 IGF-II, human; IGF-I analogue; IGF I (24-41); IGF I (57-70); IGF I (30-41); IGF II; IGF II (33-40); [Tyr⁰]-IGF II (33-40); liver cell growth factor; midkine; midkine 60-121, human; N-acetyl, alpha-TGF 34-43, methyl ester, rat; nerve growth factor (NGF), mouse; platelet-derived growth factor; platelet-derived growth factor antagonist; transforming growth factor-alpha, human; and transforming growth factor-I, rat.

Growth hormone peptides including, but not limited to, growth hormone (hGH), human; growth hormone 1-43, human; growth hormone 6-13, human; growth hormone releasing factor, human; growth hormone releasing factor, bovine; growth hormone releasing factor, porcine; growth hormone releasing factor 1-29, amide, rat; growth hormone pro-releasing factor, human; biotinyl-growth hormone releasing factor, human; growth hormone releasing factor 1-29, amide, human; [D-Ala²]-growth hormone releasing factor 1-29, amide, human; [N-Ac-Tyr¹, D-Arg²]-GRF 1-29, amide; [His¹, Nle²⁷]-growth hormone releasing factor 1-32, amide; growth hormone releasing factor 1-37, human; growth hormone releasing factor 1-40, human; growth hormone releasing factor 1-40, amide, human; growth hormone releasing factor 30-44, amide, human; growth hormone releasing factor, mouse; growth hormone releasing factor, ovine; growth hormone releasing factor, rat, biotinyl-growth hormone releasing factor, rat; GHRP-6 ([His¹, Lys⁶]-GHRP); hexarelin (growth hormone releasing hexapeptide); and [D-Lys³]-GHRP-6.

GTP-binding protein fragment peptides including, but not limited to, [Arg⁸]-GTP-binding protein fragment, Gs alpha; GTP-binding protein fragment, G beta; GTP-binding protein fragment, GAlpha; GTP-binding protein fragment, Go Alpha; GTP-binding protein fragment, Gs Alpha; and GTP-binding protein fragment, G Alpha i2.

Guanylin peptides including, but not limited to, guanylin, human; guanylin, rat; and uroguanylin.

Inhibin peptides including, but not limited to, inhibin, bovine; inhibin, alpha-subunit 1-32, human; [Tyr⁰]-inhibin, alpha-subunit 1-32, human; seminal plasma inhibin-like peptide, human; [Tyr⁰]-seminal plasma inhibin-like peptide, human; inhibin, alpha-subunit 1-32, porcine; and [Tyr⁰]-inhibin, alpha-subunit 1-32, porcine.

Insulin peptides including, but not limited to, insulin, human; insulin, porcine; IGF-I, human; insulin-like growth factor II (69-84); pro-insulin-like growth factor II (68-102), human; pro-insulin-like growth factor II (105-128), human; [AspB28]-insulin, human; LysB28]-insulin, human; [LeuB28]-insulin, human; [ValB28]-insulin, human; [AlaB28]-insulin, human; [AspB28, ProB29]-insulin, human; [LysB28, ProB29]-insulin, human; [LeuB28, ProB29]-insulin, human; [ValB28, ProB29]-insulin, human; and [AlaB28, ProB29]-insulin, human, B22-B30 insulin, human; B23-B30 insulin, human; B25-B30 insulin, human; B26-B30 insulin, human; B27-B30 insulin, human; B29-B30 insulin, human; the A chain of human insulin, and the B chain of human insulin.

Interleukin peptides including, but not limited to, interleukin-1 beta 165-181, rat; and interleukin-8 (IL-8, CINC/gro), rat.

Laminin peptides including, but not limited to, laminin; alpha 1 (I)-CB3 435-438, rat; and laminin binding inhibitor.

Leptin peptides including, but not limited to, leptin 93-105, human; leptin 22-56, rat; Tyr-leptin 26-39, human; and leptin 116-130, amide, mouse.

Leucokinin peptides including, but not limited to, leucomyosuppressin (LMS); leucopyrokinin (LPK); leucokinin I; leucokinin II; leucokinin III; leucokinin IV; leucokinin VI; leucokinin VII; and leucokinin VIII.

Luteinizing hormone-releasing hormone peptides including, but not limited to, antide; Gn-RH II, chicken; luteinizing hormone-releasing hormone (LH-RH) (GnRH); biotinyl-LH-RH; cetrorelix (D-20761); [D-Ala⁶]-LH-RH; [Gln⁸]-LH-RH (Chicken LH-RH); [DLeu⁶,Val⁷] LH-RH 1-9, ethyl amide; [D-Lys⁶]-LH-RH; [D-Phe², Pro³, D-Phe⁶]-LH-RH; [DPhe², DAla⁶] LH-RH; [Des-Gly¹⁰]-LH-RH, ethyl amide; [D-Ala⁶, Des-Gly¹⁰]-LH-RH, ethyl amide; [DTrp]-LH-RH, ethyl amide; [D-Trp, Des-Gly¹⁰]-LH-RH, ethyl amide (Deslorelin); [DSer(But)⁶, Des-Gly¹⁰]-LH-RH, ethyl amide; ethyl amide; leuprolide; LH-RH 4-10; LH-RH 7-10; LH-RH, free acid; LH-RH, lamprey; LH-RH, salmon; [Lys⁸]-LH-RH; [Trp⁷,Leu⁸] LH-RH, free acid; and [(t-Bu)DSer⁶, (Aza)Gly¹⁰]-LH-RH.

Mastoparan peptides including, but not limited to, mastoparan; mas7; mas8; mas17; and mastoparan X.

Mast cell degranulating peptides including, but not limited to, mast cell degranulating peptide HR-1; and mast cell degranulating peptide HR-2.

Melanocyte stimulating hormone (MSH) peptides including, but not limited to, [Ac-Cys⁴,DPhe⁷,Cys¹⁰] alpha-MSH 4-13, amide; alpha-melanocyte stimulating hormone; alpha-MSH, free acid; beta-MSH; porcine; biotinyl-alpha-melanocyte stimulating hormone; biotinyl-[Nle⁴, D-Phe⁷] alpha-melanocyte stimulating hormone; [Des-Acetyl]-alpha-MSH; [DPhe⁷]-alpha-MSH, amide; gamma-1-MSH, amide; [Lys⁰]-gamma-1-MSH, amide; MSH release inhibiting factor, amide; [Nle⁴]-alpha-MSH, amide; [Nle⁴,D-Phe⁷]-alpha-MSH; N-Acetyl, [Nle⁴,DPhe⁷] alpha-MSH 4-10, amide; beta-MSH, human; and gamma-MSH. Morphiceptin peptides including, but not limited to, morphiceptin (beta-casomorphin 1-4 amide); [D-Pro⁴]-morphiceptin; and [N-MePhe³,D-Pro⁴]-morphiceptin.

Motilin peptides including, but not limited to, motilin, canine; motilin, porcine; biotinyl-motilin, porcine; and [Leu¹³]-motilin, porcine.

Neuro-peptides including, but not limited to, Ac-Asp-Glu; achatina cardio-excitatory peptide-1 (ACEP-1) (Achatina fulica); adipokinetic hormone (AKH) (Locust); adipokinetic hormone (Heliothis zea and Manduca sexta); alytesin; Tabanus atratus adipokinetic hormone (Taa-AKH); adipokinetic hormone II (Locusta migratoria); adipokinetic hormone II (Schistocera gregaria); adipokinetic hormone III (AKH-3); adipokinetic hormone G (AKH-G) (Gryllus bimaculatus); allatotropin (AT) (Manduca sexta); allatotropin 6-13 (Manduca sexta); APGW amide (Lymnaea stagnalis); buccalin; cerebellin; [Des-Ser¹]-cerebellin; corazonin (American Cockroach Periplaneta americana); crustacean cardioactive peptide (CCAP); crustacean erythrophore; DF2 (Procambarus clarkii); diazepam-binding inhibitor fragment, human; diazepam binding inhibitor fragment (ODN); eledoisin related peptide; FMRF amide (molluscan cardioexcitatory neuro-peptide); Gly-Pro-Glu (GPE), human; granuliberin R; head activator neuropeptide; [His⁷]-corazonin; stick insect hypertrehalosaemic factor II; Tabanus atratus hypotrehalosemic hormone (Taa-HoTH); isoguvacine hydrochloride; bicuculline methiodide; piperidine-4-sulphonic acid; joining peptide of proopiomelanocortin (POMC), bovine; joining peptide, rat; KSAYMRF amide (P. redivivus); kassinin; kinetensin; levitide; litorin; LUQ 81-91 (Aplysia californica); LUQ 83-91 (Aplysia californica); myoactive peptide I (Periplanetin CC-1) (Neuro-hormone D); myoactive peptide II (Periplanetin CC-2); myomodulin; neuron specific peptide; neuron specific enolase 404-443, rat; neuropeptide FF; neuropeptide K, porcine; NEI (prepro-MCH 131-143) neuropeptide, rat; NGE (prepro-MCH 110-128) neuropeptide, rat; NF1 (Procambarus clarkii); PBAN-1 (Bombyx mori); Hez-PBAN (Heliothis zea); SCPB (cardioactive peptide from aplysia); secretoneurin, rat; uperolein; urechistachykinin I; urechistachykinin II; xenopsin-related peptide I; xenopsin-related peptide II; pedal peptide (Pep), aplysia; peptide F1, lobster; phyllomedusin; polistes mastoparan; proctolin; ranatensin; Ro I (Lubber Grasshopper, Romalea microptera); Ro II (Lubber Grasshopper, Romalea microptera); SALMF amide 1 (S1); SALMF amide 2 (S2); and SCPA.

Neuropeptide Y (NPY) peptides including, but not limited to, [Leu³¹,Pro³⁴]-neuropeptide Y, human; neuropeptide F (Moniezia expansa); B1BP3226 NPY antagonist; Bis (31/31′) {[Cys³¹, TrP³², Nva³⁴] NPY 31-36}; neuropeptide Y, human, rat; neuropeptide Y 1-24 amide, human; biotinyl-neuropeptide Y; [D-Tyr^{27,36}, D-Thr³²]-NPY 27-36; Des 10-17 (cyclo 7-21) [Cys^{7,21}, Pro³⁴]-NPY; C2-NPY; [Leu³¹, Pro³⁴] neuropeptide Y, human; neuropeptide Y, free acid, human; neuropeptide Y, free acid, porcine; prepro NPY 68-97, human; N-acetyl-[Leu²⁸, Leu³¹] NPY 24-36; neuropeptide Y, porcine; [D-TrP³²]-neuropeptide Y, porcine; [D-TrP³²] NPY 1-36, human; [Leu⁷,DTrp³²] neuropeptide Y, human; [Leu³¹, Pro³⁴]-NPY, porcine; NPY 2-36, porcine; NPY 3-36, human; NPY 3-36, porcine; NPY 13-36, human; NPY 13-36, porcine; NPY 16-36. porcine; NPY 18-36, porcine; NPY 20-36; NFY 22-36; NPY 26-36; [Pro³⁴]-NPY 1-36, human; [Pro³⁴]-neuropeptide Y, porcine; PYX-1; PYX-2; T4-[NPY(33-36)]4; and Tyr(OMe)²¹]-neuropeptide Y, human.

Neurotropic factor peptides including, but not limited to, glial derived neurotropic factor (GDNF); brain derived neurotropic factor (BDNF); and ciliary neurotropic factor (CNTF).

Orexin peptides including, but not limited to, orexin A; orexin B, human; orexin B, rat, mouse.

Opioid peptides including, but not limited to, alpha-casein fragment 90-95; BAM-18P; casomokinin L; casoxin D; crystalline; DALDA; dermenkephalin (deltorphin) (Phylomedusa sauvagei); [D-Ala²]-deltorphin I; [D-Ala²]-deltorphin II; endomorphin-1; endomorphin-2; kyotorphin; [DArg²]-kyotorphin; morphin tolerance peptide; morphine modulating peptide, C-terminal fragment; morphine modulating neuropeptide (A-18-F-NH2); nociceptin [orphanin FQ] (ORL1 agonist); TIPP; Tyr-MIF-1; Tyr-W-MIF-1; valorphin; LW-hemorphin-6, human; Leu-valorphin-Arg; and Z-Pro-D-Leu.

Oxytocin peptides including, but not limited to, [Asu⁶]-oxytocin; oxytocin; biotinyl-oxytocin; [Thr⁴, Gly⁷]-oxytocin; and tocinoic acid ([Ile³]-pressinoic acid).

PACAP (pituitary adenylating cyclase activating peptide) peptides including, but not limited to, PACAP 1-27, human, ovine, rat; PACAP (1-27)-Gly-Lys-Arg-NH2, human; [Des-G¹⁶]-PACAP 6-27, human, ovine, rat; PACAP38, frog; PACAP27-NH2, human, ovine, rat; biotinyl-PACAP 27-NH2, human, ovine, rat; PACAP 6-27, human, ovine, rat; PACAP38, human, ovine, rat; biotinyl-PACAP38, human, ovine, rat; PACAP 6-38, human, ovine, rat; PACAP 27-NH2, human, ovine, rat; biotinyl-PACAP 27-NH2, human, ovine, rat; PACAP 6-27, human, ovine, rat; PACAP38, human, ovine, rat; biotinyl-PACAP 38, human, ovine, rat; PACAP 6-38, human, ovine, rat; PACAP38 16-38, human, ovine, rat; PACAP 38 31-38, human, ovine, rat; PACAP38 31-38, human, ovine, rat; PACAP-related peptide (PRP), human; and PACAP-related peptide (PRP), rat.

Pancreastatin peptides including, but not limited to, chromostatin, bovine; pancreastatin (hPST-52) (chromogranin A 250-301, amide); pancreastatin 24-52 (hPST-29), human; chromogranin A 286-301, amide, human; pancreastatin, porcine; biotinyl-pancreastatin, porcine; [Nle⁸]-pancreastatin, porcine; [Tyr⁰,Nle⁸]-pancreastatin, porcine; [Tyr⁰]-pancreastatin, porcine; parastatin 1-19 (chromogranin A 347-365), porcine; pancreastatin (chromogranin A 264-314-amide, rat; biotinyl-pancreastatin (biotinyl-chromogranin A 264-314-amide; [Tyr⁰]-pancreastatin, rat; pancreastatin 26-51, rat; and pancreastatin 33-49, porcine.

Pancreatic polypeptides including, but not limited to, pancreatic polypeptide, avian; pancreatic polypeptide, human; C-fragment pancreatic polypeptide acid, human; C-fragment pancreatic polypeptide amide, human; pancreatic polypeptide (Rana temporaria); pancreatic polypeptide, rat; and pancreatic polypeptide, salmon.

Parathyroid hormone peptides including, but not limited to, [Asp⁷⁶]-parathyroid hormone 39-84, human; [Asp⁷⁶]-parathyroid hormone 53-84, human; [Asp⁷⁶]-parathyroid hormone 1-84, hormone; [Asp⁷⁶]-parathyroid hormone 64-84, human; [Asn⁸, Leu¹⁸]-parathyroid hormone 1-34, human; [Cys⁵′²⁸]-parathyroid hormone 1-34, human; hypercalcemia malignancy factor 1-40; [Leu¹⁸]-parathyroid hormone 1-34, human; [Lys(biotinyl)¹³, Nle^{8,18}, Tyr³⁴]-parathyroid hormone 1-34 amide; [Nle^{8,18}, Tyr³⁴]-parathyroid hormone 1-34 amide; [Nle^{8,18}, Tyr³⁴]-parathyroid hormone 3-34 amide, bovine; [Nle^{8,18}, Tyr³⁴]-parathyroid hormone 1-34, human; [Nle^{8,18}, Tyr³⁴]-parathyroid hormone 1-34 amide, human; [Nle^{8,18}, Tyr³⁴]-parathyroid hormone 3-34 amide, human; [Nle^{8,18}, Tyr³⁴]-parathyroid hormone 7-34 amide, bovine; [Nle^{8,18}, Tyr³⁴]-parathyroid hormone 1-34 amide, rat; parathyroid hormone 44-68, human; parathyroid hormone 1-34, bovine; parathyroid hormone 3-34, bovine; parathyroid hormone 1-31 amide, human; parathyroid hormone 1-34, human; parathyroid hormone 13-34, human;-parathyroid hormone 1-34, rat; parathyroid hormone 1-38, human; parathyroid hormone 1-44, human; parathyroid hormone 28-48, human; parathyroid hormone 39-68, human; parathyroid hormone 39-84, human; parathyroid hormone 53-84, human; parathyroid hormone 69-84, human; parathyroid hormone 70-84, human; [Pro³⁴]-peptide YY (PYY), human; [Tyr⁰]-hypercalcemia malignancy factor 1-40; [Tyr⁰]-parathyroid hormone 1-44, human; [Tyr⁰]-parathyroid hormone 1-34, human; [Tyr¹]-parathyroid hormone 1-34, human; [Tyr²⁷]-parathyroid hormone 27-48, human; [Tyr³⁴]-parathyroid hormone 7-34 amide, bovine; [Tyr⁴³]-parathyroid hormone 43-68, human; [Tyr⁵², Asn⁷⁶]-parathyroid hormone 52-84, human; and [Tyr⁶³]-parathyroid hormone 63-84, human.

Parathyroid hormone (PTH)-related peptides including, but not limited to, PTHrP ([Tyr³⁶]-PTHrP 1-36 amide), chicken; hHCF-(1-34)-NH2 (humoral hypercalcemic factor), human; PTH-related protein 1-34, human; biotinyl-PTH-related protein 1-34, human; [Tyr⁰]-PTH-related protein 1-34, human; [Tyr³⁴]-PTH-related protein 1-34 amide, human; PTH-related protein 1-37, human; PTH-related protein 7-34 amide, human; PTH-related protein 38-64 amide, human; PTH-related protein 67-86 amide, human; PTH-related protein 107-111, human, rat, mouse; PTH-related protein 107-111 free acid; PTH-related protein 107-138, human; and PTH-related protein 109-111, human.

Peptide T peptides including, but not limited to, peptide T; [D-Ala¹]-peptide T; and [D-Ala¹]-peptide T amide.

Prolactin-releasing peptides including, but not limited to, prolactin-releasing peptide 31, human; prolactin-releasing peptide 20, human; prolactin-releasing peptide 31, rat; prolactin-releasing peptide 20, rat; prolactin-releasing peptide 31, bovine; and prolactin-releasing peptide 20, bovine.

Peptide YY (PYY) peptides including, but not limited to, PYY, human; PYY 3-36, human; biotinyl-PYY, human; PYY, porcine, rat; and [Leu³¹, Pro³⁴]-PYY, human.

Renin substrate peptides including, but not limited to, acetyl, angiotensinogen 1-14, human; angiotensinogen 1-14, porcine; renin substrate tetradecapeptide, rat; [Cys⁸]-renin substrate tetradecapeptide, rat; [Leu⁸]-renin substrate tetradecapeptide, rat; and [Val⁸]-renin substrate tetradecapeptide, rat.

Secretin peptides including, but not limited to, secretin, canine; secretin, chicken; secretin, human; biotinyl-secretin, human; secretin, porcine; and secretin, rat.

Somatostatin (GIF) peptides including, but not limited to, BIM-23027; biotinyl-somatostatin; biotinylated cortistatin 17, human; cortistatin 14, rat; cortistatin 17, human; [Tyr⁰]-cortistatin 17, human; cortistatin 29, rat; [D-Trp⁸]-somatostatin; [DTrp⁸,DCys¹⁴]-somatostatin; [DTrp⁸,Tyr¹¹]-somatostatin; [D-Trp¹¹]-somatostatin; NTB (Naltriben); [Nle⁸]-somatostatin 1-28; octreotide (SMS 201-995); prosomatostatin 1-32, porcine; [Tyr⁰]-somatostatin; [Tyr¹]-somatostatin; [Tyr¹]-somatostatin 28 (1-14); [Tyr¹¹]-somatostatin; [Tyr⁰, D-Trp⁸]-somatostatin; somatostatin; somatostatin antagonist; somatostatin-25; somatostatin-28; somatostatin 28 (1-12); biotinyl-somatostatin-28; [Tyr⁰]-somatostatin-28; [Leu⁸,D-Trp²², Tyr²⁵]-somatostatin-28; biotinyl-[Leu⁸,D-Trp²²,Tyr²⁵]-somatostatin-28; somatostatin-28 (1-14); and somatostatin analog, RC-160.

Substance P peptides including, but not limited to, G protein antagonist-2; Ac-[Arg⁶, Sar⁹, Met(02)¹¹]-substance P 6-11; [Arg³]-substance P; Ac-Trp-3,5-bis(trifluoromethyl) benzyl ester; Ac-[Arg⁶, Sar⁹, Met(O2)¹¹]-substance P 6-11; [D-Ala⁴]-substance P 4-11; [Tyr⁶, D-Phe⁷, D-His⁹]-substance P 6-11. (sendide); biotinyl-substance P; biotinyl-NTE[Arg³]-substance P; [Tyr⁸]-substance P; [Sar⁹, Met(O2)¹¹]-substance P; [D-Pro², D-Trp^{7,9}]-substance P; [D-Pro⁴, 0-Trp^{7,9}]-substance P 4-11; substance P 4-11; [DTrp^{2,7,9}]-substance P; [(Dehydro)Pro^{2,4}, Pro⁹]-substance P; [Dehydro-Pro⁴]-substance P 4-11; [Glp⁵,(Me)Phe⁸, Sar⁹]-substance P 5-11; [Glp⁵,Sar⁹]-substance P 5-11; [Glp⁵]-substance P 5-11; hepta-substance P (substance P 5-11); hexa-substance P (substance P 6-11); [MePhe⁸,Sar⁹]-substance P; [Nle¹¹]-substance P; Octa-substance P (substance P 4-11); [pGlu¹]-hexa-substance P ([pGlu⁶]-substance P 6-11); [pGlu⁶, D-Pro⁹]-substance P 6-11; [(pNO2)Phe⁷Nle¹¹]-substance P; penta-substance P (substance P 7-11); [Pro⁹]-substance P; GR73632, substance P 7-11 [Sar⁴]-substance P 4-11; [Sar⁹]-substance P; septide ([pGlu⁶, Pro9]-substance P 6-11); spantide I; spantide II; substance P; substance P, cod; substance P, trout; substance P antagonist; substance P-Gly-Lys-Arg; substance P-1-4; substance P 1-6; substance P 1-7; substance P 1-9; deca-substance P (substance P 2-11); nova-substance P (substance P 3-11); substance P tetrapeptide (substance P 8-11); substance P tripeptide (substance P 9-11); substance P, free acid; substance P methyl ester; and [Tyr⁸,Nle¹¹] substance P.

Tachykinin peptides including, but not limited to, [Ala⁵, beta-Ala⁸] neurokinin A 4-10; eledoisin; locustatachykinin I (Lom-TK-I) (Locusta migratoria); locustatachykinin II (Lom-TK-II) (Locusta migratoria); neurokinin A 4-10; neurokinin A (neuromedin L, substance K); neurokinin A, cod and trout; biotinyl-neurokinin A (biotinyl-neuromedin L, biotinyl-substance K); [Tyr⁰]-neurokinin A; [Tyr⁶]-substance K; [Lys³, Gly⁸-(R)-gamma-lactam-Leu⁹]-neurokinin A 3-10; [Beta-Ala₈]-neurokinin A 4-10; [Nle¹⁰]-neurokinin A 4-10; [Trp⁷, beta-Ala⁸]-neurokinin A 4-10; neurokinin B (neuromedin K); biotinyl-neurokinin B (biotinyl-neuromedin K); [MePhe⁷]-neurokinin B; [Pro⁷]-neurokinin B; [Tyr⁰]-neurokinin B; neuromedin B, porcine; biotinyl-neuromedin B, porcine; neuromedin B-30, porcine; neuromedin B-32, porcine; neuromedin B receptor antagonist; neuromedin C, porcine; neuromedin N, porcine; neuromedin (U-8), porcine; neuromedin (U-25), porcine; neuromedin U, rat; neuropeptide-gamma (gamma-preprotachykinin 72-92); PG-KII; phyllolitorin; [Leu⁸]-phyllolitorin (Phyllomedusa sauvagei); physalaemin; physalaemin 1-11; scyliorhinin II, amide, dogfish; senktide, selective neurokinin B receptor peptide; [Ser2]-neuromedin C; beta-preprotachykinin 69-91, human; beta-preprotachykinin 111-129, human; tachyplesin I; xenopsin; and xenopsin 25 (xenin 25), human.

Thyrotropin-releasing hormone (TRH) peptides including, but not limited to, biotinyl-thyrotropin-releasing hormone; [Glu¹]-TRH; His-Pro-diketopiperazine; [3-Me-His²]-TRH; pGlu-Gln-Pro-amide; pGlu-His; [Phe²]-TRH; prepro TRH 53-74; prepro TRH 83-106; prepro-TRH 160-169 (Ps4, TRH-potentiating peptide); prepro-TRH 178-199; thyrotropin-releasing hormone (TRH); TRH, free acid; TRH-SH Pro; and TRH precursor peptide.

Vasoactive intestinal peptides (VIP/PHI) including, but not limited to, VIP, human, porcine, rat, ovine; VIP-Gly-Lys-Arg-NH2; biotinyl-PHI (biotinyl-PHI-27),-porcine; [Glp¹⁶] VIP 16-28, porcine; PHI (PHI-27), porcine; PHI (PHI-27), rat; PHM-27 (PHI), human; prepro VIP 81-122, human; preproVIP/PHM 111-122; prepro VIP/PHM 156-170; biotinyl-PHM-27 (biotinyl-PHI), human; vasoactive intestinal contractor (endothelin-beta); vasoactive intestinal octacosa-peptide, chicken; vasoactive intestinal peptide, guinea pig; biotinyl-VIP, human, porcine, rat; vasoactive intestinal peptide 1-12, human, porcine, rat; vasoactive intestinal peptide 10-28, human, porcine, rat; vasoactive intestinal peptide 11-28, human, porcine, rat, ovine; vasoactive intestinal peptide (cod, Gadus morhua); vasoactive intestinal peptide 6-28; vasoactive intestinal peptide antagonist; vasoactive intestinal peptide antagonist ([Ac-Tyr¹, D-Phe²]-GHRF 1-29 amide); vasoactive intestinal peptide receptor antagonist (4-C1-D-Phe⁶, Leu¹⁷]-VIP); and vasoactive intestinal peptide receptor binding inhibitor, L-8-K.

Vasopressin (ADH) peptides including, but not limited to, vasopressin; [Asu^{1,6},Arg⁸]-vasopressin; vasotocin; [Asu^{1,6},Arg⁸]-vasotocin; [Lys⁸]-vasopressin; pressinoic acid; [Arg⁸]-desamino vasopressin desglycinamide; [Arg⁸]-vasopressin (AVP); [Arg⁸]-vasopressin desglycinamide; biotinyl-[Arg⁸]-vasopressin (biotinyl-AVP); [D-Arg⁸]-vasopressin; desamino-[Arg⁸]-vasopressin; desamino-[D-Arg⁸]-vasopressin (DDAVP); [deamino-[D-3-(3′-pyridyl-Ala)]-[Arg⁸]-vasopressin; [1-(beta-Mercapto-beta, beta-cyclopentamethylene propionic acid), 2-(O-methyl)tyrosine]-[Arg⁸]-vasopressin; vasopressin metabolite neuropeptide [pGlu⁴, Cys⁶]; vasopressin metabolite neuropeptide [pGlu⁴, Cys6]; [Lys]-deamino vasopressin desglycinamide; [Lys⁸]-vasopressin; [Mpr¹,Val⁴,D-Arg⁸]-vasopressin; [Phe², Ile³, Orn⁸]-vasopressin ([Phe², Orn⁸]-vasotocin); [Arg⁸]-vasotocin; and [d(CH₂)₅, Tyr(Me)², Orn⁸]-vasotocin.

While certain analogs, fragments; and/or analog fragments of the various polypeptides have been described above, it is to be understood that other analogs, fragments, and/or analog fragments that retain all or some of the activity of the particular polypeptide may also be useful in embodiments of the present invention. Analogs may be obtained by various means, as will be understood by those skilled in the art. For example, certain amino acids may be substituted for other amino acids in a polypeptide without appreciable loss of interactive binding capacity with structures such as, for example, antigen-binding regions of antibodies or binding sites on substrate molecules. As the interactive capacity and nature of a polypeptide drug defines its biological functional activity, certain amino acid sequence substitutions can be made in the amino acid sequence and nevertheless remain a polypeptide with like properties.

It is also understood in the art that the substitution of like amino acids can be made effectively on a number of different basis, one of which is hydrophilicity. U.S. Pat. No. 4,554,101 provides that the greatest local average hydrophilicity of a protein, as governed by the hydrophilicity of its adjacent amino acids, correlates with a biological property of the protein. As detailed in U.S. Pat. No. 4,554,101, the following hydrophilicity values have been assigned to amino acid residues: arginine (+3.0); lysine (+−.3.0); aspartate (+3.0.+-0.1); glutamate (+3.0.+-0.1); serine (+0.3); asparagine (+0.2); glutamine (+0.2); glycine (0); threonine (−0.4); proline (−0.5.+-0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). As is understood by those skilled in the art, an amino acid can be substituted for another having a similar hydrophilicity value and still obtain a biologically equivalent, and in particular, an immunologically equivalent polypeptide. In such changes, the substitution of amino acids whose hydrophilicity values are within +−0.2 of each other is preferred, those which are within +−0.1 of each other are generally more preferred, and those within +−.0.5 of each other are better.

As noted herein, amino acid substitutions are generally therefore based on the relative similarity of the amino acid side-chain substituents, for example, their hydrophobicity, hydrophilicity, charge, size, and the like. Exemplary substitutions (i.e., amino acids that may be interchanged without significantly altering the biological activity of the polypeptide) that take various of the foregoing characteristics into consideration are well known to those of skill in the art and include, for example: arginine and lysine; glutamate and aspartate; serine and threonine; glutamine and asparagine; and valine, leucine and isoleucine.

When the protein is calcitonin, and more particularly salmon calcitonin, the CPS may be coupled to an amino functionality of the salmon calcitonin, including the amino functionality of Lys 11, Lys 18 and the N-terminus. It is recognized, that similar to insulin, one or more CPS peptides (and each CPS peptide may have differing repeat sequences within them, etc. as explained above) may be coupled to the protein, such as on the amino functionality of Lys 11 and Lys 18.

When the protein is human growth hormone, the CPS peptide may be coupled to an amino functionality of Phe1, Lys 38, Lys 41, Lys 70, Lys 115, Lys 140, Lys 145, Lys 158, Lys 168, and/or Lys 172. Again, the protein may have one or more CPS peptide chains attached, and the CPS peptide may each independently range from 11 to about 50 amino acids with repeating 11 mer units as defined herein.

It may be desirable to obtain differential conjugation at particular sites on the protein, and/or to obtain particular mixtures of the protein—CPS conjugate. Conjugation of the peptide at the amino functionality of lysine in a protein may be suppressed by maintaining the pH of the reaction solution below the pKa of lysine. Mixtures of the protein-CPS conjugate may be separated and isolated utilizing, for example HPLC to provide the desired mixture of mono, di or tri-conjugates. The degree of conjugation (e.g., whether the isolated molecule is a mono-, di- or tri-conjugate) of a particular isolated conjugate-protein complex may be determined and/or verified utilizing various techniques as will be understood by those skilled in the art including, but not limited to mass spectroscopy. A particular structure may be determined or verified utilizing various techniques as will be understood by those skilled in the art including, but not limited to, sequence analysis, peptide mapping, selective enzymatic cleavage, and/or endopeptidase cleavage.

As noted, the CPS peptide may be coupled to the protein or polypeptide where a nucleophilic hydroxyl or amino function is found. For example a nucleophilic hydroxyl function may be a serine and/or tyrosine residue; a nucleophilic amino function may be a histidine, and/or a lysine residue, and/or one or more N-termini of the polypeptide. When the CPS is coupled to one or more N-termini of the polypeptide, the coupling may form a secondary amine. For instance, when the polypeptide is insulin, the CPS can be coupled to the amino functionality of Gly A1, the amino functionality of Phe B1, or Lys B29.

In addition to blocking reaction sites on the CPS peptide in order to couple the peptide to the protein, as will be understood by those skilled in the art, it may also be desirable to block one or more of the reaction sites on the protein or polypeptide. For example the polypeptide may be reacted with a suitable blocking reagent such as N-tert-butoxycarbonyl (t-BOC) or, N-(9-fluoroenylmethoxycarbonyl) (N-FMOC). Following such blocking, the mixture of blocked polypeptide, and blocked and activated CPS peptide may be reacted to provide the desired conjugates. After the conjugation reaction, the peptide-protein conjugates may be de-blocked as will be understood by those skilled in the art. If necessary they may then be separated in to mixtures, or separate into mixture prior to de-blocking.

The following are definitions of the terms as used throughout this specification and claims. The definitions provided apply throughout the present specification unless otherwise indicated. Terms not defined herein have the meaning commonly understood in the art to which the term pertains.

“Addition” when used in reference to an amino acid sequence, includes extensions of one or more amino acids at either or both ends of the sequence as well as insertions within the sequence.

“Conservative” used in reference to an addition, deletion or substitution of an amino acid means an addition, deletion or substitution in an amino acid chain that does not completely diminish the therapeutic efficacy of the peptide. For example, in an insulin compound the efficacy may be reduced, the same, or enhanced, relative to the therapeutic efficacy of scientifically acceptable control, such as a corresponding native insulin compound.

“Hydrophilic” means exhibiting characteristics of water solubility, and the term “hydrophilic moiety” refers to a moiety which is hydrophilic and/or which when attached to another chemical entity, increases the hydrophilicity of such chemical entity.

“Lipophilic” means exhibiting characteristics of fat solubility, such as accumulation in fat and fatty tissues, the ability to dissolve in lipids and/or the ability to penetrate, interact with and/or traverse biological membranes, and the term, “lipophilic moiety” means a moiety which is lipophilic and/or which, when attached to another chemical entity, increases the lipophilicity of such chemical entity.

“Proinsulin compound” means an insulin compound in which the C-terminus of the B-chain is coupled to the N-terminus of the A-chain via a natural or artificial C-peptide having 5 or more amino acids.

“Preproinsulin compound” means a proinsulin compound further including a leader sequence coupled to the N-terminus of the B-chain, such as a sequence selected to promote excretion as a soluble protein, or a sequence selected to prevent conjugation of the N-terminus, or a sequence selected to enhance purification (e.g., a sequence with binding affinity to a purification column).

“Single chain insulin compound precursor” or “miniproinsulin compound” means an insulin compound in which the C-terminus of the B-chain (or a truncated B-chain having 1, 2, 3 or 4 amino acids removed from the C-terminus) is coupled to the N-terminus of the A-chain or a truncated A-chain shortened at the N-terminus by 1, 2, 3 or 4 amino acids, without an intervening C-peptide, or via a shortened C-peptide having 1, 2, 3 or 4 amino acids.

“Protamine” refers to a mixture of strongly basic proteins obtained from natural (e.g., fish sperm) or recombinant sources. See Hoffmann, J. A., et al., Protein Expression and Purification, 1:127-133 (1990). The protamine composition can be provided in a relatively salt-free preparation of the proteins, often called “protamine base” or in a preparation including salts of the proteins.

“Protein”, “peptide” and “polypeptide” are used interchangeably herein to refer to compounds having amino acid sequences of at least two and up to any length.

“Substitution” means replacement of one or more amino acid residues within a sequence of amino acids with another amino acid. In some cases, the substituted amino acid acts as a functional equivalent, resulting in a silent alteration. Substitutions may be conservative; for example, conservative substitutions may be selected from other members of the class to which the substituted amino acid belongs. Examples of non-polar (hydrophobic) amino acids include alanine, leucine, isoleucine, valine, proline, phenylalanine, tryptophan and methionine. Examples of polar neutral amino acids include glycine, serine, threonine, cysteine, tyrosine, asparagine, and glutamine. Examples of positively charged (basic) amino acids include arginine, lysine and histidine. Examples of negatively charged (acidic) amino acids include aspartic acid and glutamic acid.

“Water solubility” or “aqueous solubility” unless otherwise indicated, is determined in an aqueous buffer solution at a pH of 7.4.

The CPS peptides of the present invention can be prepared by standard peptide synthesis methods known to those of skill in the art. The CPS peptides may also be produced using an expression vector having a nucleotide sequence encoding the peptide(s) of choice operably linked to appropriate promoter, terminator, and other functional sequences, such as a sequence encoding a purification tag, to facilitate expression and purification of the peptides. “Operably” or “functionally” linked means that the CPS and its peptide, e.g. insulin are connected so that the CPS can direct import of the CPS/peptide (e.g., insulin conjugate) into the cell and the insulin, or other suitable peptide, referred to as a cargo peptide in U.S. Ser. No. 11/270,295 can function to affect cellular metabolism, such as cell signaling as desired. As noted above, CPS and the polypeptide (or cargo peptide as it is referred to U.S. Ser. No. 11/270,295) can be linked, for example, by one or more peptide bonds. The CPS can be immediately C-terminal or N-terminal to the cargo peptide, and more than one CPS can be used, more than one cargo peptide can be used, and/or the CPS and cargo peptide amino acid sequences can be separated by one or more amino acids in the region between the CPS and cargo peptide. The CPS/cargo peptide can comprise additional amino acids either C-terminal or N-terminal, or both.

The CPS/cargo peptides may be formulated for administration in a pharmaceutical carrier in accordance with known techniques. See, e.g., Alfonso R. Gennaro, Remington: The Science and Practice of Pharmacy, Lippincott Williams & Wilkins Publishers (June 2003), and Howard C. Ansel, Pharmaceutical Dosage Forms and Drug Delivery Systems, Lippincott Williams & Wilkins Publishers, 7th ed. (October 1999), the entire disclosures of which are incorporated herein by reference for their teachings concerning the selection, making and using of pharmaceutical dosage forms.

The carrier used herein must be acceptable in the sense of being compatible with any other ingredients in the pharmaceutical composition and should not be unduly deleterious to the subject, relative to the benefit provided by the active ingredient(s). The carrier may be a solid or a liquid, or both. It is preferably formulated as a unit-dose formulation, for example, a tablet. The pharmaceutical compositions may be prepared by any of the well known techniques of pharmacy including, but not limited to, admixing the components, optionally including one or more accessory ingredients.

Examples of suitable pharmaceutical compositions include those made for oral, rectal, inhalation (e.g., via an aerosol) buccal (e.g., sub-lingual), vaginal, parenteral (e.g., subcutaneous, intramuscular, intradermal, intraarticular, intrapleural, intraperitoneal, intracerebral, intra-arterial, or intravenous), topical, mucosal surfaces (including airway surfaces), nasal surfaces, and transdermal administration. The most suitable route in any given case will depend on the nature and severity of the condition being treated and on the nature of the particular insulin conjugate being used. Oral compositions are compositions prepared for ingestion by the subject. Ideally, the oral compositions are prepared to survive or substantially survive passage through the stomach and to completely or substantially completely dissolve in the intestine for delivery of the active ingredient. Examples of suitable transdermal systems include ultrasonic, iontophoretic, and patch delivery systems. Inhalation is also a suitable means for delivery.

Pharmaceutical compositions suitable for oral administration may be presented in discrete units, such as capsules, cachets, lozenges, or tables, each containing a predetermined amount of the mixture of insulin compound conjugates; as a powder or granules; as a solution or a suspension in an aqueous or non-aqueous liquid; or as an oil-in-water or water-in-oil emulsion. Such formulations may be prepared by any suitable method of pharmacy which includes the step of bringing into association the mixture of conjugates and a suitable carrier (which may contain one or more accessory ingredients as noted above). Formulations may include suspensions of solids, insulin conjugates, active ingredient (e.g., native insulin compound, insulin compound conjugates), and/or mixtures of the foregoing.

In general, the pharmaceutical compositions of the invention are prepared by uniformly and intimately admixing the complexes with a liquid or solid carrier, or both, and then, if necessary, shaping the resulting mixture. For example, a tablet may be prepared by compressing or molding a powder or granules containing the mixture of insulin compound conjugates, optionally with one or more accessory ingredients. Compressed tablets may be prepared by compressing, in a suitable machine, the mixture in a free-flowing form, such as a powder or granules optionally mixed with a binder, lubricant, inert diluent, and/or surface active/dispersing agent(s). Molded tablets may be made by molding, in a suitable machine, the powdered composition moistened with an inert liquid binder.

Pharmaceutical compositions suitable for buccal (sub-lingual) administration include lozenges comprising the mixture of insulin conjugates in a flavored base, such as sucrose and acacia or tragacanth; and pastilles comprising the mixture of insulin conjugate in an inert base such as gelatin and glycerin or sucrose and acacia. For pulmonary delivery of insulin formulations, see U.S. Pat. No. 6,737,045 (“Methods and compositions for the pulmonary delivery insulin compound”); U.S. Pat. No. 6,730,334 (“Multi-arm block copolymers as drug delivery vehicles”); U.S. Pat. No. 6,685,967 (“Methods and compositions for pulmonary delivery of insulin compound”); U.S. Pat. No. 6,630,169 (“Particulate delivery systems and methods of use”); U.S. Pat. No. 6,589,560 (“Stable glassy state powder formulations; U.S. Pat. No. 6,592,904 (“Dispersible macromolecule compositions and methods for their preparation and use”); U.S. Pat. No. 6,582,728 (“Spray drying of macromolecules to produce inhalable dry powders”); U.S. Pat. No. 6,565,885 (“Methods of spray drying pharmaceutical compositions”); U.S. Pat. No. 6,546,929 (“Dry powder dispersing apparatus and methods for their use”); U.S. Pat. No. 6,543,448 (“Apparatus and methods for dispersing dry powder medicaments”); U.S. Pat. No. 6,518,239 (“Dry powder compositions having improved dispersivity”); U.S. Pat. No. 6,514,496 (“Dispersible antibody compositions and methods for their preparation and use”); U.S. Pat. No. 6,509,006 (“Devices compositions and methods for the pulmonary delivery of aerosolized medicaments”); U.S. Pat. No. 6,433,040 (“Stabilized bioactive preparations and methods of use”); U.S. Pat. No. 6,423,344 (“Dispersible macromolecule compositions and methods for their preparation and use”); U.S. Pat. No. 6,372,258 (“Methods of spray-drying a drug and a hydrophobic amino acid”); U.S. Pat. No. 6,309,671 (“Stable glassy state powder formulations”); U.S. Pat. No. 6,309,623 (“Stabilized preparations for use in metered dose inhalers”); U.S. Pat. No. 6,294,204 (“Method of producing morphologically uniform microcapsules and microcapsules produced by this method”); U.S. Pat. No. 6,267,155 (“Powder filling systems, apparatus and methods”); U.S. Pat. No. 6,258,341 (“Stable glassy state powder formulations”); U.S. Pat. No. 6,182,712 (“Power filling apparatus and methods for their use”); U.S. Pat. No. 6,165,463 (“Dispersible antibody compositions and methods for their preparation and use”); U.S. Pat. No. 6,138,668 (“Method and device for delivering aerosolized medicaments”); U.S. Pat. No. 6,103,270 (“Methods and system for processing dispersible fine powders”); U.S. Pat. No. 6,089,228 (“Apparatus and methods for dispersing dry powder medicaments”); U.S. Pat. No. 6,080,721 (“Pulmonary delivery of active fragments of parathyroid hormone”); U.S. Pat. No. 6,051,256 (“Dispersible macromolecule compositions and methods for their preparation and use”); U.S. Pat. No. 6,019,968 (“Dispersible antibody compositions and methods for their preparation and use”); U.S. Pat. No. 5,997,848 (“Methods and compositions for pulmonary delivery of insulin compound”); U.S. Pat. No. 5,993,783 (“Method and apparatus for pulmonary administration of dry powder.alpha.1-antitrypsin”); U.S. Pat. No. 5,922,354 (“Methods and system for processing dispersible fine powders”); U.S. Pat. No. 5,826,633 (“Powder filling systems, apparatus and methods”); U.S. Pat. No. 5,814,607 (“Pulmonary delivery of active fragments of parathyroid hormone”); U.S. Pat. No. 5,785,049 (“Method and apparatus for dispersion of dry powder medicaments”); U.S. Pat. No. 5,780,014 (“Method and apparatus for pulmonary administration of dry powder alpha 1-antitrypsin”); U.S. Pat. No. 5,775,320 (“Method and device for delivering aerosolized medicaments”); U.S. Pat. No. 5,740,794 (“Apparatus and methods for dispersing dry powder medicaments”); U.S. Pat. No. 5,654,007 (“Methods and system for processing dispersible fine powders”); U.S. Pat. No. 5,607,915 (“Pulmonary delivery of active fragments of parathyroid hormone”); U.S. Pat. No. 5,458,135 (“Method and device for delivering aerosolized medicaments”); U.S. Pat. No. 6,602,952 (“Hydrogels derived from chitosan and poly(ethylene glycol) or related polymers”); and U.S. Pat. No. 5,932,462 (“Multiarmed, monofunctional, polymer for coupling to molecules and surfaces”).

In one embodiment of the present invention, the agents of the present invention are delivered via oral inhalation or intranasal administration. Appropriate dosage forms for such administration, such as an aerosol formulation or a metered dose inhaler, may be prepared by conventional techniques.

For administration by inhalation the compounds may be delivered in the form of an aerosol spray presentation from pressurized packs or a nebulizer, with the use of a suitable propellant, e.g. dichlorodifluoromethane, trichlorofluoromethane, dichlorotetrafluoroethane, a hydrofluoroalkane such as tetrafluoroethane or heptafluoropropane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of a compound of the invention and a suitable powder base such as lactose or starch.

Dry powder compositions for topical delivery to the lung by inhalation may, for example, be presented in capsules and cartridges, for example gelatin; or blisters, for example laminated aluminum foil, for use in an inhaler or insufflator. Powder blend formulations generally contain a powder mix for inhalation of the compound of the invention and a suitable powder base (carrier/diluent/excipient substance) such as mono-, di- or poly-sacchamides (e.g. lactose or starch).

Each capsule or cartridge may generally contain between 20 μg-10 mg of the conjugate, and optionally in combination with another therapeutically active ingredient. Alternatively, the conjugate or the protein and CPS peptide as individual agents, may be presented without excipients.

Suitably, the packing/medicament dispenser is of a type selected from the group consisting of a reservoir dry powder inhaler (RDPI), a multi-dose dry powder inhaler (MDPI), and a metered dose inhaler (MDI).

By reservoir dry powder inhaler (RDPI) it is meant an inhaler having a reservoir form pack suitable for comprising multiple (un-metered doses) of medicament in dry powder form and including means for metering medicament dose from the reservoir to a delivery position. The metering means may for example comprise a metering cup, which is movable from a first position where the cup may be filled with medicament from the reservoir to a second position where the metered medicament dose is made available to the patient for inhalation.

By multi-dose dry powder inhaler (MDPI) is meant an inhaler suitable for dispensing medicament in dry powder form, wherein the medicament is comprised within a multi-dose pack containing (or otherwise carrying) multiple, define doses (or parts thereof) of medicament. In a preferred aspect, the carrier has a blister pack form, but it could also, for example, comprise a capsule-based pack form or a carrier onto which medicament has been applied by any suitable process including printing, painting and vacuum occlusion.

In the case of multi-dose delivery, the formulation can be pre-metered (e.g. as in Diskus, see GB 2242134, U.S. Pat. Nos. 6,632,666, 5,860,419, 5,873,360 and 5,590,645 or Diskhaler, see GB 2178965, 2129691 and 2169265, U.S. Pat. Nos. 4,778,054, 4,811,731, 5,035,237, the disclosures of which are hereby incorporated by reference) or metered in use (e.g. as in Turbuhaler, see EP 69715 or in the devices described in U.S. Pat. No. 6,321,747 the disclosures of which are hereby incorporated by reference). An example of a unit-dose device is Rotahaler (see GB 2064336 and U.S. Pat. No. 4,353,656, the disclosures of which are hereby incorporated by reference).

The Diskus inhalation device comprises an elongate strip formed from a base sheet having a plurality of recesses spaced along its length and a lid sheet hermetically but peelably sealed thereto to define a plurality of containers, each container having therein an inhalable formulation containing a conjugate. Preferably, the strip is sufficiently flexible to be wound into a roll. The lid sheet and base sheet will preferably have leading end portions which are not sealed to one another and at least one of the said leading end portions is constructed to be attached to a winding means. Also, preferably the hermetic seal between the base and lid sheets extends over their whole width. The lid sheet may preferably be peeled from the base sheet in a longitudinal direction from a first end of the said base sheet.

In one aspect, the multi-dose pack is a blister pack comprising multiple blisters for containment of medicament in dry powder form. The blisters are typically arranged in regular fashion for ease of release of medicament there from.

In one aspect, the multi-dose blister pack comprises plural blisters arranged in generally circular fashion on a disc-form blister pack. In another aspect, the multi-dose blister pack is elongate in form, for example comprising a strip or a tape.

In one aspect, the multi-dose blister pack is defined between two members peelably secured to one another. U.S. Pat. Nos. 5,860,419, 5,873,360 and 5,590,645 describe medicament packs of this general type. In this aspect, the device is usually provided with an opening station comprising peeling means for peeling the members apart to access each medicament dose. Suitably, the device is adapted for use where the peelable members are elongate sheets which define a plurality of medicament containers spaced along the length thereof, the device being provided with indexing means for indexing each container in turn. More preferably, the device is adapted for use where one of the sheets is a base sheet having a plurality of pockets therein, and the other of the sheets is a lid sheet, each pocket and the adjacent part of the lid sheet defining a respective one of the containers, the device comprising driving means for pulling the lid sheet and base sheet apart at the opening station.

By metered dose inhaler (MDI) it is meant a medicament dispenser suitable for dispensing medicament in aerosol form, wherein the medicament is comprised in an aerosol container suitable for containing a propellant-based aerosol medicament formulation. The aerosol container is typically provided with a metering valve, for example a slide valve, for release of the aerosol form medicament formulation to the patient. The aerosol container is generally designed to deliver a predetermined dose of medicament upon each actuation by means of the valve, which can be opened either by depressing the valve while the container is held stationary or by depressing the container while the valve is held stationary.

Where the medicament container is an aerosol container, the valve typically comprises a valve body having an inlet port through which a medicament aerosol formulation may enter said valve body, an outlet port through which the aerosol may exit the valve body and an open/close mechanism by means of which flow through said outlet port is controllable.

The valve may be a slide valve wherein the open/close mechanism comprises a sealing ring and receivable by the sealing ring a valve stem having a dispensing passage, the valve stem being slidably movable within the ring from a valve-closed to a valve-open position in which the interior of the valve body is in communication with the exterior of the valve body via the dispensing passage.

Typically, the valve is a metering valve. The metering volumes are typically from 10 to 100 μl, such as 25 μl, 50 μl or 63 μl. Suitably, the valve body defines a metering chamber for metering an amount of medicament formulation and an open/close mechanism by means of which the flow through the inlet port to the metering chamber is controllable. Preferably, the valve body has a sampling chamber in communication with the metering chamber via a second inlet port, said inlet port being controllable by means of an open/close mechanism thereby regulating the flow of medicament formulation into the metering chamber.

The valve may also comprise a ‘free flow aerosol valve’ having a chamber and a valve stem extending into the chamber and movable relative to the chamber between dispensing and non-dispensing positions. The valve stem has a configuration and the chamber has an internal configuration such that a metered volume is defined there between and such that during movement between is non-dispensing and dispensing positions the valve stem sequentially: (i) allows free flow of aerosol formulation into the chamber, (ii) defines a closed metered volume for pressurized aerosol formulation between the external surface of the valve stem and internal surface of the chamber, and (iii) moves with the closed metered volume within the chamber without decreasing the volume of the closed metered volume until the metered volume communicates with an outlet passage thereby allowing dispensing of the metered volume of pressurized aerosol formulation. A valve of this type is described in U.S. Pat. No. 5,772,085. Additionally, intra-nasal delivery of the present compounds is effective.

To formulate an effective pharmaceutical nasal composition, the medicament must be delivered readily to all portions of the nasal cavities (the target tissues) where it performs its pharmacological function. Additionally, the medicament should remain in contact with the target tissues for relatively long periods of time. The longer the medicament remains in contact with the target tissues, the medicament must be capable of resisting those forces in the nasal passages that function to remove particles from the nose. Such forces, referred to as ‘mucociliary clearance’, are recognized as being extremely effective in removing particles from the nose in a rapid manner, for example, within 10-30 minutes from the time the particles enter the nose.

Other desired characteristics of a nasal composition are that it must not contain ingredients which cause the user discomfort, that it has satisfactory stability and shelf-life properties, and that it does not include constituents that are considered to be detrimental to the environment, for example ozone depletors.

A suitable dosing regime for the formulation of the present invention when administered to the nose would be for the patient to inhale deeply subsequent to the nasal cavity being cleared. During inhalation the formulation would be applied to one nostril while the other is manually compressed. This procedure would then be repeated for the other nostril.

One means for applying the formulation of the present invention to the nasal passages is by use of a pre-compression pump. Most preferably, the pre-compression pump will be a VP7 model manufactured by Valois SA. Such a pump is beneficial as it will ensure that the formulation is not released until a sufficient force has been applied, otherwise smaller doses may be applied. Another advantage of the pre-compression pump is that atomisation of the spray is ensured as it will not release the formulation until the threshold pressure for effectively atomising the spray has been achieved. Typically, the VP7 model may be used with a bottle capable of holding 10-50 ml of a formulation. Each spray will typically deliver 50-100 μl of such a formulation; therefore, the VP7 model is capable of providing at least 100 metered doses.

Spray compositions for topical delivery to the lung by inhalation may for example be formulated as aqueous solutions or suspensions or as aerosols delivered from pressurized packs, such as a metered dose inhaler, with the use of a suitable liquefied propellant. Aerosol compositions suitable for inhalation can be either a suspension or a solution and generally contain the conjugate or the protein along with the CPS peptide, optionally in combination with another therapeutically active ingredient, and a suitable propellant such as a fluorocarbon or hydrogen-containing chlorofluorocarbon or mixtures thereof, particularly hydrofluoroalkanes, e.g. dichlorodifluoromethane, trichlorofluoromethane, dichlorotetra-fluoroethane, especially 1,1,1,2-tetrafluoroethane, 1,1,1,2,3,3,3-heptafluoro-n-propane or a mixture thereof. Carbon dioxide or other suitable gas may also be used as propellant. The aerosol composition may be excipient free or may optionally contain additional formulation excipients well known in the art such as surfactants, e.g., oleic acid or lecithin and cosolvents, e.g. ethanol. Pressurized formulations will generally be retained in a canister (e.g. an aluminum canister) closed with a valve (e.g. a metering valve) and fitted into an actuator provided with a mouthpiece.

Medicaments for administration by inhalation desirably have a controlled particle size. The optimum particle size for inhalation into the bronchial system is usually 1-10 μm, preferably 2-5 μm. Particles having a size above 20 μm are generally too large when inhaled to reach the small airways. To achieve these particle sizes the particles of the active ingredient as produced may be size reduced by conventional means e.g., by micronization. The desired fraction may be separated out by air classification or sieving. Suitably, the particles will be crystalline in form. When an excipient such as lactose is employed, generally, the particle size of the excipient will be much greater than the inhaled medicament within the present invention. When the excipient is lactose it will typically be present as milled lactose, wherein not more than 85% of lactose particles will have a MMD of 60-90 μm and not less than 15% will have a MMD of less than 15 μm.

Intranasal sprays may be formulated with aqueous or non-aqueous vehicles with the addition of agents such as thickening agents, buffer salts or acid or alkali to adjust the pH, isotonicity adjusting agents or anti-oxidants.

Solutions for inhalation by nebulization may be formulated with an aqueous vehicle with the addition of agents such as acid or alkali, buffer salts, isotonicity adjusting agents or antimicrobials. They may be sterilised by filtration or heating in an autoclave, or presented as a non-sterile product.

Suitably, administration by inhalation may preferably target the organ of interest for respiratory diseases, i.e. the lung, and in doing so may reduce the efficacious dose needed to be delivered to the patient. In addition, administration by inhalation may reduce the systemic exposure of the compound thus avoiding effects of the compound outside the lung.

Pharmaceutical compositions according to embodiments of the invention suitable for parenteral administration comprise sterile aqueous and non-aqueous injection solutions of the complexes, which preparations are preferably isotonic with the blood of the intended recipient. These preparations may contain anti-oxidants, buffers, bacteriostats and solutes which render the composition isotonic with the blood of the intended recipient. Aqueous and non-aqueous sterile suspensions may include suspending agents and thickening agents. The compositions may be presented in unit\dose or multi-dose containers, for example sealed ampoules and vials, and may be stored in a freeze-dried (lyophilized) condition requiring only the addition of the sterile liquid carrier, for example, saline or water-for-injection immediately prior to use. Extemporaneous injection solutions and suspensions may be prepared from sterile powders, granules and tablets of the kind previously described. For example, an injectable, stable, sterile composition with a mixture of complexes in a unit dosage form in a sealed container may be provided. The mixture of complexes can be provided in the form of a lyophilizate which is capable of being reconstituted with a suitable pharmaceutically acceptable carrier to form a liquid composition suitable for injection into a subject. The parenteral unit dosage form typically comprises from about 1 microgram to about 10 mg of the mixture of insulin conjugate. When the complexes are substantially water-insoluble, a sufficient amount of emulsifying agent which is physiologically acceptable may be employed in sufficient quantity to emulsify the complexes in an aqueous carrier. One such useful emulsifying agent is phosphatidyl choline.

A solid dosage form for oral administration typically includes from about 2 mg to about 500 mg, preferably about 10 mg to about 250 mg, ideally about 20 mg to about 110 mg of the insulin conjugate.

Pharmaceutical compositions suitable for rectal administration are suitably presented as unit dose suppository. These may be prepared by admixing the insulin conjugate with one or more conventional solid carriers, for example, cocoa butter, and then shaping the resulting mixture. Pharmaceutical compositions suitable for topical application to the skin preferably take the form of an ointment, cream, lotion, paste, gel, spray, aerosol, or oil. Carriers which may be used include petroleum jelly, lanoline, PEG's, alcohols, transdermal enhancers, and combinations of two or more thereof.

The insulin conjugate compositions and formulations thereof are useful in the treatment of conditions in which increasing the amount of insulin compound (relative to the amount provided by the subject in the absence of administration of insulin compound from an exogenous source) into to the cell, provides for or yields a desirable therapeutic or physiological effect. For example, the condition treated may be Type I or Type II diabetes, prediabetes and/or metabolic syndrome. In one embodiment, the compositions are administered to alleviate symptoms of diabetes. In another embodiment, the compositions are administered to a prediabetic subject in order to prevent or delay the onset of diabetes.

The effective amount of the insulin conjugate composition for administration according to the methods of the invention will vary somewhat from mixture to mixture, and subject to subject, and will depend upon factors such as the age and condition of the subject, the route of delivery and the condition being treated. Such dosages can be determined in accordance with routine pharmacological procedures known to those skilled in the art.

As a general proposition, an oral dosage from about 0.025 to about 10 mg/kg of active ingredient (i.e., the conjugate) will have therapeutic efficacy, with all weights being calculated based upon the weight of the mixture of insulin conjugates. In one embodiment the oral dose is about 0.06 to about 1 mg/kg.

A parenteral dosage typically ranges from about 0.5 .mcg/kg to about 0.5 mg/kg, with all weights being calculated based upon the weight of the mixture of insulin compound conjugates. In one embodiment of the invention, the parenteral dosage of a peptide conjugate is from about 1 mcg/kg to about 100 mcg/kg.

The frequency of administration is usually one, two, or three times per day or as necessary to control the condition. The duration of treatment depends on the type of insulin compound deficiency being treated and may be for as long as the life of the subject. The conjugates may, for example, be administered within 0 to 30 minutes prior to a meal. The conjugates may, for example, be administered within 0 to 2 hours prior to bedtime.

Cell permeable, “importation competent” signal peptide sequences, and membrane translocation sequences facilitate the transport of attached peptides and proteins into cells. Several sequences of this kind have previously been described, including the hydrophobic region of the signal sequence of Kaposi fibroblast growth factor which has been fused to the nuclear localization sequence (NLS) of p50 to produce the peptide known as SN50. The novel CPS sequence confers improved cell permeability with an attached polypeptide or protein. It is believed that operably linking a polypeptide such as insulin to a cell permeable sequence (CPS) of Lys-Leu-Lys-Leu-Ala-Leu-Ala-Leu-Ala-Leu-Ala (SEQ ID No. 1) will produce a peptide having improved activity when compared to the activity of insulin under similar conditions of administration.

As used herein, the term “CPS” includes variants or biologically active fragments of the peptides sequence SEQ ID No. 1, as well as peptides which may contain additional amino acids either N-terminal or C-terminal (or both) to the disclosed sequences, their derivatives, variants, or functional counterparts. A “functional counterpart” can include, for example, a peptide nucleic acid (PNA). A “variant” of the peptide is not completely identical to a disclosed CPS peptide sequence. A variant, given the disclosure of the present invention, can be obtained by altering the amino acid sequence by insertion, deletion or substitution of one or more amino acid. The amino acid sequence of a disclosed peptide can be modified, for example, by substitution to create a peptide having substantially the same or improved qualities. The substitution may be a conserved substitution. A “conserved substitution” is a substitution of an amino acid with another amino acid having a side chain that is similar in polar/nonpolar nature, charge, or size. The 20 essential amino acids can be grouped as those having nonpolar side chains (alanine, valine, leucine, isoleucine, proline, phenylalanine, and tryptophan), uncharged polar side chains (methionine; glycine, serine, threonine, cysteine, tyrosine, asparagine and glutamine), acidic side chains (aspartate and glutamate) and basic side chains (lysine, arginine, and histidine). Conserved substitutions might include, for example, Asp to Glu, Asn or Gln; His to Lys, Arg or Phe; Asn to Gln, Asp or Glu, Leu to Ile or Val, and Ser to Cys, Thr or Gly. Alanine is commonly used to make conserved substitutions.

To those of skill in the art, variant peptides can be obtained by substituting a first amino acid for a second amino acid at one or more positions in the peptide structure in order to affect biological activity. Amino acid substitutions may, for example, induce conformational changes in a polypeptide that result in increased biological activity. Those of skill in the art may also make substitutions in the amino acid sequence based on the hydrophilicity index or hydropathic index of the amino acids.

A variant peptide of the present invention has less than 100%, but at least about 50%, and more preferably at least about 80% to about 90% amino acid sequence homology or identity to the amino acid sequence of a corresponding native nucleic acid molecule or polypeptide comprising SEQ ID NO 1. The amino acid sequence of a variant CPS peptide therefore corresponds essentially to the disclosed amino acid sequences. As used herein, “corresponds essentially to” refers to a polypeptide sequence that will elicit a similar biological activity as that generated by the disclosed CPS, such activity being from at least about 70 percent of that of disclosed CPS peptide, to greater than 100 percent of the activity of a disclosed CPS peptide.

A variant of a disclosed CPS may include amino acid residues not present in the corresponding CPS, or may include deletions relative to the corresponding CPS. A variant may also be a truncated “fragment” as compared to the corresponding CPS, i.e., only a portion of the amino acid sequence of the CPS peptide.

The cell permeable sequences of the present invention can be used to deliver a variety of other peptides, nucleic acids, and other organic compounds for research or therapeutic use as noted herein. In addition to those peptides already mentioned that can be delivered to the interior of the cell using the method of the present invention include, but are not limited to, peptides that comprise enzyme cleavage sites, phosphorylation sites, protein-protein interaction regions, and receptor binding sites of intracellular proteins.

It is believed that the increased membrane permeability of the CPS peptide will provide for a more effective agent for delivering the active agent, comprising, for example, a peptide, protein, DNS, RNA, antisense oligonucleotide, ribozyme, or combination thereof, through one or more tissues to aid in drug delivery.

The CPS peptide/insulin conjugate can be achieved in several ways, such as by total chemical synthesis of human insulin, e.g., chemical synthesis of A-chain, B-chain, and CPS peptide; purification; denaturation, re-naturation, and oxidation of the conjugate. Alternatively, one could conjugate the CPS molecule to commercially available insulin, such as by synthesis of CPS peptide-NHS activated ester, or by direct conjugation of CPS peptide to the insulin moiety as described earlier. Suitably the peptide is first converted to an active form for reactivity with the desired amino acid on the larger protein. This is accomplished by chemical means, such as using the carboxyl group on the C-terminus of the CPS peptide. The carboxyl group may be activated using N-hydroxysulfosuccinimide (Sulfo-NHS) or its uncharged analog, N-hydroxysuccinimide (NHS). The Sulfo-NHS is reacted by mixing with the CPS peptide and a suitably dehydrating agent, such as carbodiimide-EDC (EDAC) to yield the amine-reactive Sulfo-NHS esters. Alternative activating agent carbodiimides include but are not limited to Dicyclohexylcarbodiimide (DCC), diisopropylcarbodiimide (DIPCDI or DIC), t-butylmethylcarbodiimide, carbonyldiimidazole, HATU, and t-butylethylcarbodiimide. A principal limitation in using carbodiimides is the dehydration of Asn and Gln residues. Addition of HoBt to the mixture may prevent dehydration and has an added benefit of acting as a catalyst. A suitable textbook is J. Stewart et al., Solid Phase Peptide Synthesis, 2^{nd} Ed., Pierce Chemical (1984) although later texts are more highly recommended for use on Fmoc procedures.

Efficient peptide-bond formation requires chemical activation of the carboxyl component of the N-alpha protected amino acid. The activating group or reaction must be carefully chosen to achieve a very high coupling efficiency and at the same time avoid potential side reactions. In situ activating agents are widely accepted because they are easy to use, give fast reactions, and generally free of side reactions. Most are based on phosphonium or aminium (uronium) salts in the presence of a tertiary base, and can smoothly convert protected amino acids to a variety of activated species as desired. Most commonly employed are BOP, PyBOP, HBTU, and TBTU. Having successfully synthesized a protected peptide on a resin, the detachment of the peptide and removal of the side chain protecting groups generally takes place. In the instance of the CPS peptide it may be desirable to retain the protected amino acid derivatives until conjugation with the polypeptide, and or small molecule has occurred. It is recognized that the skilled artisan will need to make appropriate choices for the protected amino acid derivative, and resin. Use of TFA/TIS/water may generally suffice for most sequences but will at the discretion of the skilled artisan. Addition of EDT, as a scavenger reagent, may be added if desired.

Suitable N-alpha-Fmoc protected amino acids used as building blocks in solid phase synthesis, as well as standard N-alpha Boc protected amino acids, and other amino acid derivatives are well known in the art. A number of them may be found for purchase at Novabiochem, EMD BioScience, Inc., California. In particular, an Fmoc-Lys(Boc)-OH is recommended for the routine preparation of lysine containing peptides. For the preparation of cyclic peptide and peptide containing side-chain modified Lys residues, derivatives such as Fmoc-Lys(Mtt)-OH, Fmoc-Lys(ivDde(-OH) should be used side their respective side chain protecting groups can be removed selectively on the solid phase. In the coupling reaction of the insulin compound (or polypeptide) to the activated CPS peptide, will likely occur in the presence of a base, such as diisopropyl ethylamine, or triethylamine.

The invention will be further described by means of the following non-limiting examples.

### Design and Synthesis of CPS Functional Peptides

The CPS peptide is synthesized by conventional solid-phase peptide synthesis methodology (Celtek Bioscience, Nashville, Tenn.). Standard synthesis protocols based on Fmoc chemistry were used. After synthesis, the crude peptides are cleaved from the solid support and purified by C₁₈ reverse-phase HPLC. The purified peptides are characterized by analytical HPLC analysis and mass spectrometry analysis.

To the CPS protein is added N-hydroxysulfosuccinimide (Sulfo-NHS) or its uncharged analog N-hydroxysuccinimide (NHS). A suitable dehydrating agent EDC, will react with the carboxyl group at the C-terminal of the peptide, forming an amine-reactive O-acylisourea intermediate. This intermediate will react with the amine on B29-LYS of insulin (or the 2 other N-terminus A1, B1) under suitable conditions, yielding a conjugate of the two molecules joined by a stable amide bond. The chemical intermediate is susceptible to hydrolysis, making it unstable and short-lived in aqueous solution. Therefore, the addition of Sulfo-NHS (5 mM) stabilizes the amine-reactive intermediate by converting it to an amine-reactive Sulfo-NHS ester, increasing the efficiency of EDC-mediated coupling reactions. The amine-reactive Sulfo-NHS ester intermediate has sufficient stability to permit the necessary two-step crosslinking procedures, which allows the carboxyl groups on insulin to remain unaltered.

### Examples of Synthesis

Initial synthesis was of the 11-mer CPS peptide in which its C-terminal end was an N-hydroxysuccinimide (NHS) ester. This CPS-OSu peptide was expected to be highly reactive to the amine groups of human insulin and thus facilitate the conjugation reactions between CPS and Insulin. It was determined that the 11-mer peptide was transformed to its NHS ester derivative, and it became highly unstable as it tended to react with its own amine groups in the N-terminal region internally. As a result, this CPS-OSu activated ester was cyclized via an internal amide bond. To overcome this side-reaction, a derivative of the CPS-OSu peptide in which all three amine groups of this peptide were protected by a Boc group (tert-Butyloxycarbonyl) was also made. This amine-protected peptide showed a poor solubility in aqueous solutions to be purified by HPLC methods.

To avoid the head-to-tail internal amide bond cyclization of the CPS-OSu activated ester as discussed above, the design of the CPS peptide was modified by including a Cys residue at the C-terminus. By such a setting, a commercially available hetero-bifunctional reagent could be used such as the m-Maleimidobenzoyl-N-hydroxysuccinimide ester (Sulfo-MBS, Pierce). This small MBS cross-linker is both amine-reactive and sulfhydryl-reactive and thus can link CPS-Cys to insulin via a two-staged reaction as described below.

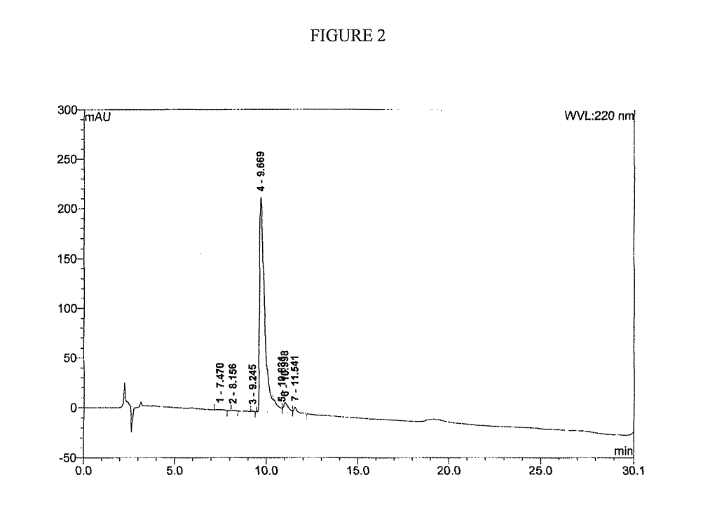

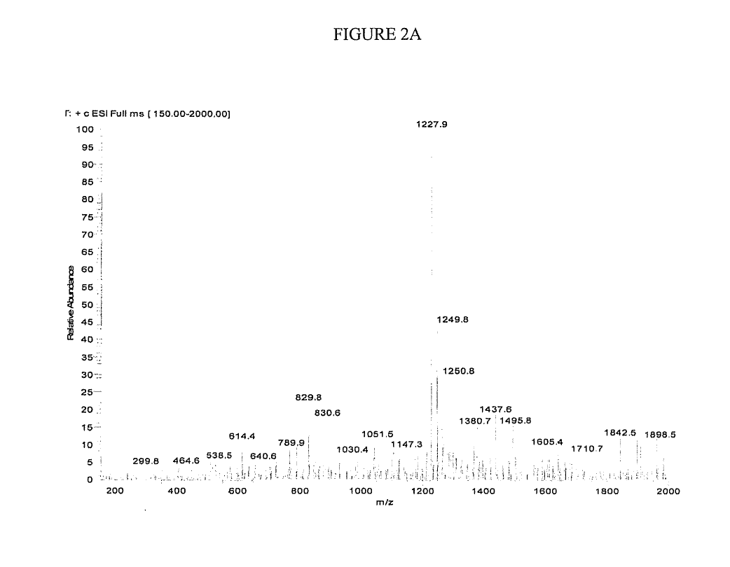

The 12-mer CPS-Cys peptide Sequence of CPS-Cys Peptide: K-L-K-L-A-L-A-L-A-L-A-C (Seq ID no. 2) was synthesized by conventional solid-phase peptide synthesis methodology. Standard synthesis protocols based on Fmoc chemistry were used. After synthesis, the crude peptides were cleaved from the solid support and purified by C₁₈ reverse-phase HPLC eluted with acetonitrile aqueous buffers. The purified peptide was characterized by analytical HPLC analysis (FIG. 2, top panel) and mass spectrometry analysis (FIG. 2a, bottom panel, calculated MW=1227.6 Da and measured MW (MH+)=1227.9 Da). This peptide was stable and had good solubility in aqueous solutions, particularly in slightly acidic pH.

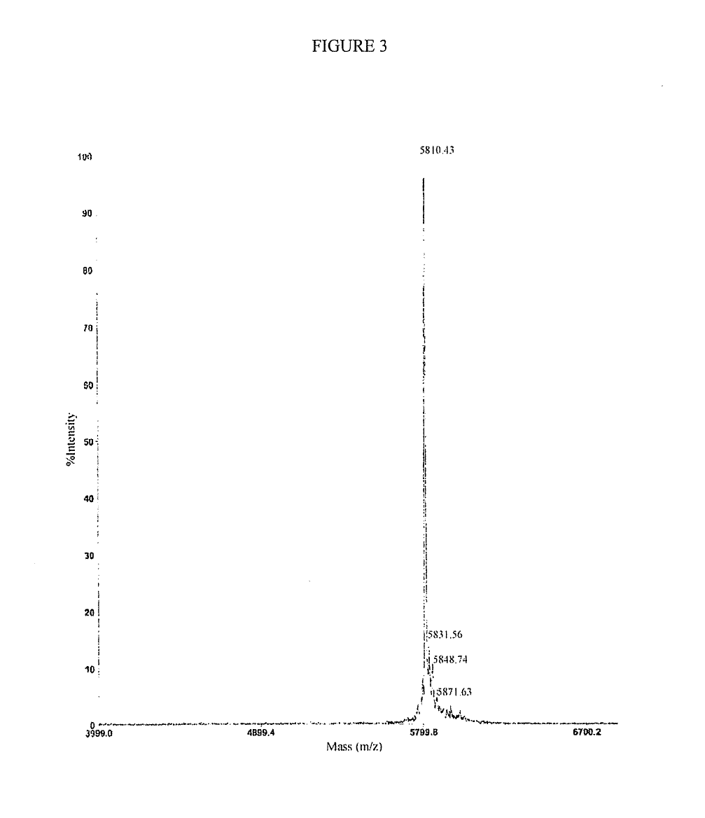

For preparing human insulin-CPS, the MBS was first conjugated (via its NHS activated ester moiety) to amine groups of human insulin according to the manufacturer's protocol, and the excess of the MBS was then removed by the dialysis. The human insulin was purchased from Serologicals Corporation (now Millipore/Upstate) and by American Peptide Company, See FIG. 3, calculated MW=5808 and measured MW (MH+)=5810), is the mass spectrometry analysis of human insulin

Briefly, MBS was added to insulin in conjugation buffer (0.1 M PBS buffer solution, pH 7.0, containing 5 mM EDTA) and the reaction kept at room temperature for 45 min before dialysis. The maleimidobenzoyl-insulin intermediate was identified by both HPLC and mass spectrometry analysis during the reaction process (calculated MW=6008, measured MW (MH+)=6010, data not shown).

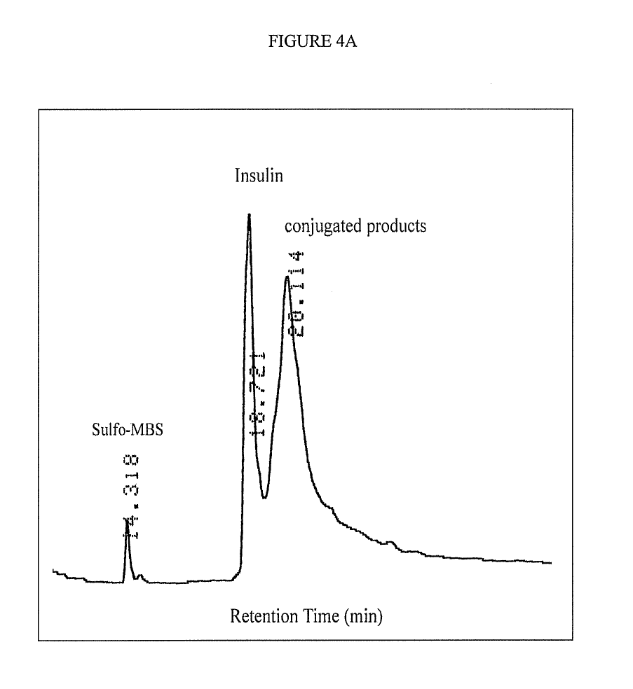

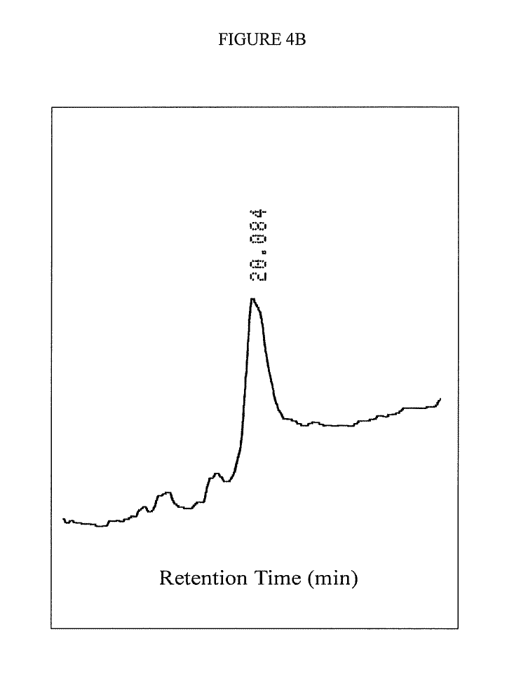

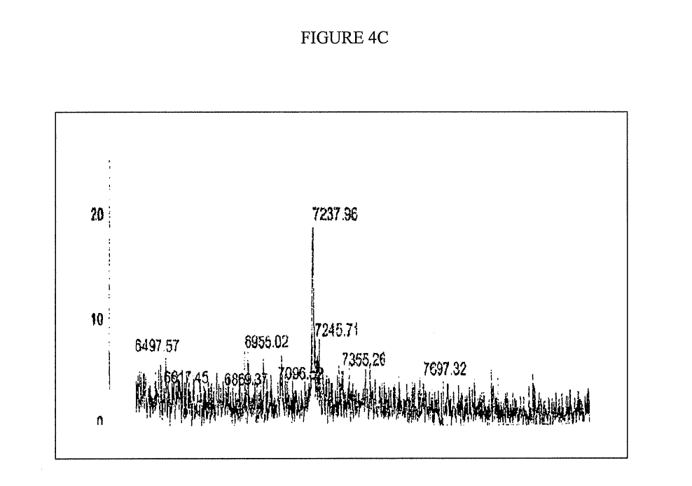

After dialysis, the maleimidobenzoyl-insulin was conjugated to CPS peptide via a high specific reaction between the maleimide group of maleimidobenzoyl-insulin and the thiol group of the Cys residue in CPS-Cys peptide. Before the conjugation, the CPS-Cys peptide was treated with the immobilized TCEP disulfide reducing gel (Pierce) for 1 hour at room temperature to assure the thiol group in Cys residue was in reducing state. After the treatment, CBS-Cys peptide was added to maleimidobenzoyl-insulin solution in conjugation buffer (0.1 M PBS buffer solution, pH 7.0, containing 5 mM EDTA) and the reaction kept at room temperature for 1-2 h. The reaction was monitored by analytical HPLC analysis. As determined by mass spectrometry analysis (MALDI), CPS-Cys was conjugated to human insulin to form Insulin-CPS (FIG. 4, calculated MW=7235 and measured MW (MH+)=7237).

FIG. 4A demonstrates the conjugation reaction of CPS-Cys peptide to insulin via Sulfo-MBS was monitored by analytical HPLC. FIG. 4B demonstrates the conjugated product Insulin-CPS purified by HPLC which shows a retention time greater than that of unconjugated insulin. FIG. 4C demonstrates the mass spectrometry analysis of the HPLC fraction (on panel B) showed a molecular mass of 7237 Da (MH+) consistent with the calculated MW of Insulin-CPS.

In order to optimizing the conditions to improve the yield and specificity of the Insulin-CPS conjugation reaction, testing smaller hetero-bifunctional reagents, such as N-succinimidyl iodoacetate (SIA, from Pierce), is used to form the link between insulin and CPS. SIA is less hydrophobic and its reaction with sulfhydryl group of CPS-Cys peptide is more specific because of its resistance to hydrolysis.

### Alternative Peptide Synthesis:

Both 11-mer CPS and 12-mer CPS-Cys (KC-12) peptides were synthesized by conventional solid-phase peptide synthesis methodology. Standard synthesis protocols based on Fmoc chemistry were used. After synthesis, the crude peptides were cleft off the solid resin support and purified by C₁₈ reverse-phase HPLC eluted with a gradient acetonitrile aqueous buffers containing 0.04% of TFA. The purified peptide was dried by lypholization and characterized by analytical HPLC analysis and mass spectrometry analysis.

Insulin Conjugated with CPS Peptide:

### Sulfo-MBS as Linker

6 mg of human insulin (American Peptide) in 6 ml of conjugation buffer (0.1 M PBS, pH 7.0, 5 mM EDTA) was incubated with 0.5 mg of sulfo-MBS (m-maleimidobenzoyl-N-hydroxysuccinimide ester, Pierce, Rockford, Ill.) in dark at RT for 30 min. On the other hand, the CPS-Cys (KC-12) peptide was treated with the immobilized TCEP disulfide reducing gel (Pierce) for 1 h at room temperature according to the manufacture protocol to assure the thiol group in Cys residue was in reducing state. For conjugation, 4 mg of KC-12 peptide in 0.7 ml of H2O was added to the reaction. The reaction mixture was kept in dark at RT for overnight. The conjugated products were subjected to C₁₈ reverse-phase HPLC analysis eluted with a linear acetonitrile gradient (1-50% for 30 min) containing 0.04% TFA and mass spectrometry analysis.

### SIA as Linker

1.5 mg of SIA (N-succinimidyl iodoacetate, Pierce, Rockford, Ill.) freshly dissolved in 0.75 ml of DMSO was added slowly with stirring to 7.5 ml of conjugation buffer (50 mM borate buffer, pH 8.3, 5 mM EDTA) containing 4 mg of human insulin (American Peptide) in dark. The reaction mixture was incubated in dark at RT for 30 min and then subjected to dialysis (MWCO 2000) with 500 ml of conjugation buffer for two times. In the meantime, the CPS-Cys (KC-12) peptide was treated with the immobilized TCEP disulfide reducing gel (Pierce) for 1 h at room temperature according to the manufacture protocol to assure the thiol group in Cys residue was in reducing state. For conjugation, 2 mg of KC-12 peptide in 8 ml of H₂O was added. The reaction mixture was kept in dark at RT for overnight, and then dialyzed with 500 ml of 5 mM PBS, pH 7.4 for four times. The conjugated products were subjected to C₁₈ reverse-phase HPLC analysis eluted with a linear acetonitrile gradient (1-50% for 30 min) containing 0.04% TFA and mass spectrometry analysis.

It is recognized that the linkers that used for conjugating CPS peptide to insulin are not limited to MBS and SIA. Other commercially available linkers can be used as long as they are chemically suitable for this type of conjugation reaction. It is also recognized that use of different linkers may improve the reaction yield of the synthesis and the solubility of the resulting CPS-insulin conjugate in aqueous solutions and buffers.

In an alternative embodiment of the invention a peptide spacer may be added between CPS and insulin to facilitate the conjugation reaction and/or increase the solubility of the resulting CPS-insulin conjugate in aqueous solutions and buffers.

In another embodiment of the invention a peptide tag, such as His tag, may be added between CPS and insulin to facilitate the purification of the CPS-insulin conjugate by an affinity-based column and/or increase the solubility of the CPS-insulin conjugate.

The experimental conditions in conjugation protocols shown above can be modified when a new peptide and/or a new linker are used in order to increase the reaction yield or conjugation specificity.

For biological assays, peptide stocks are made either in PBS (2 mg/ml) or in DMSO (30 mg/ml) as diluent. The final concentration of DMSO in the culture medium should not exceed 0.1%.

Gavin, J., Proc. Nat. Acad. Sci, USA, Vol. 71, No. 1, pp 84-88 (1974), whose disclosure is incorporated by reference herein in its entirety, describes at least one suitable cell based assay for determination of insulin—conjugate activity versus that of native insulin.

Other cell based assays, such as those which use Myeloma IM9 cells may also be used as an in vitro model. Specifically, an insulin-receptor binding competition assay will be used to determine the insulin receptor binding activity of CPS-insulin. The ability of CPS-insulin to compete with FITC-labeled insulin for receptor occupancy will be determined using a modified ELISA. In addition, the activity of CPS-insulin in inducing insulin receptor autophosphorylation will be compared with that of regular insulin without the attached CPS.

Another assay can determine whether CPS-insulin can be efficiently imported into the MDCK and Caco-2 cells in culture by using an indirect immunofluoresence assay. This assay utilizes anti-insulin antibodies which are prepared for assessing the cellular import activity of CPS-insulin. Import activity will be compared to insulin without the attached (conjugated) CPS. The level of cellular import of CPS-insulin will/can be quantified using a modified fluorescence assay. In addition, concentration, time and temperature-dependence of the cellular import of CPS-insulin will/can be evaluated. Finally, CPS-insulin will/can be examined for cytotoxicity in culture using the MTT assay according to published procedures.

### Indirect Immunofluorescence Assay for Detecting Peptide Cellular Import

DU145 cells are grown on 8-well chamber slides (Nunc, Naperville, Ill.) to a confluence of 80%. These cells are then incubated with diluent or different concentrations of peptides in RPMI without serum for 1 h at 37° C. The cells are washed three times with cold PBS to remove the extracellular peptides and then fixed with 3.5% paraformaldehyde solution in PBS at 4° C. for 20 min. The fixed cells are washed three times with cold PBS and treated with 0.25% Triton X-100 for 10 min. The washed cells are then incubated with anti-peptide IgG in PBS for 1 h. After three 5 min washings with PBS, the intracellular peptides (via peptide-antibody complexes) are subsequently detected with FITC-labeled goat anti-rabbit IgG (Pierce, Rockford, Ill.) after 1 h incubation. Cover slips with stained cells are mounted in Poly/Mount (Polysciences, Warrington, Pa.) and analyzed with Microstar IV (Reichard, Buffalo, N.Y.) using a 100× oil immersion lens. The color images are analyzed using a Pixera digital camera and stored in JPG format. The same assay may also be utilized for determining peptide cellular import in other cell lines, including PC3, LNCaP, and neuroblastoma N2a cells.

### Flow Cytometric Analysis

PCA cells were grown on 60-mm dishes to a confluence of 50-60%. These cells were incubated with different concentrations of peptides for 30 min at 37° C. followed by the treatment with TNF-α (10 ng/ml), cisplatin (2-30 μg/ml), etoposide (2-20 μg/ml) or diluent for an additional 21 h at 37° C. Phosphatidylserine exposure on apoptotic cells was measured by their ability to bind Annexin V. Specifically, cells were harvested by trypsinization. The trypsinized cells, media and PBS washes were combined and cells collected by centrifugation. The collected cells were washed with binding buffer and resuspended in 70 μl of binding buffer containing Annexin V-FITC and PI for 15 min on dark at room temperature as suggested by manufacturer's protocol (BD Biosciences, San Diego, Calif.). Stained cells are analyzed by flow cytometry. A minimum 20,000 events for each sample are preferably measured.

Alternative in vivo testing provides for use of pancreactomized and normal fasted dogs. This type of data will/can be used to show whether the orally administered conjugated insulin is absorbed, and whether it is associated with glucose lowering effects. If the insulin is absorbed in a dose dependent manner, this should also show concominant dose-dependent glucose lowering effects.

Cell based assay provide a means of confirming the biological activity (e.g., the ability of the conjugate to elicit CCK release from CCK-releasing cells) for the LCRF conjugate. Cell based assays can be used to compare the effect of treatment of the CPS-LCRF conjugate to treatment with vehicle. Native LCRF is said to elicit about a 300% increase in CCK secretion. A suitable cell based assay for LCRF can be found in WO 01/41812, Ekwuribe et al. whose disclosure is incorporated by reference herein.

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore, the Examples herein are to be construed as merely illustrative and not a limitation of the scope of the present invention in any way. The embodiments of the invention in which an exclusive property or privilege is claimed are defined as follows.

1. A peptide conjugate comprising:
a) a cell-permeable peptide of about 11 to about 50 residues comprising at least one residue of SEQ ID NO: 1 or SEQ ID No.:2; and
b) a compound selected from the group consisting of an insulin compound, calcitonin, calcitonin gene related peptide, parathyroid hormone, and luteinizing hormone-releasing hormone.

2. The peptide according to claim 1 wherein the cell-permeable peptide contains at least 2 repeating units of the 11 amino acid sequence of SEQ ID No: 1.

3. The peptide according to claim 2 wherein the cell-permeable peptide contains at least 3 repeating units of the 11 amino acid sequence of SEQ ID No: 1.

4. The peptide according to claim 3 wherein the cell-permeable peptide contains at least 4 repeating units of the 11 amino acid sequence of SEQ ID No: 1.

5. The peptide according to claim 1 wherein the repeating units of the 11 amino acid sequence of SEQ ID No: 1 or the 12 amino acid sequence of SEQ ID No.:2 are separated by at least one or amino acid residues.

6. The peptide according to claim 5 wherein the separated amino acid residue is alanine.

7. The conjugate according to claim 1 wherein the insulin is a mammalian insulin.

8. The conjugate according to claim 7 wherein the mammalian insulin is selected from the group consisting of human insulin, bovine insulin compound, and porcine insulin compound.

9. The peptide according to claim 1 which is modified N-hydrosuccinimide ester.

10. The conjugate according to claim 1 wherein the peptide is bound independently to the insulin compound at least one of the A1 N terminus, the B1 N-terminus and/or at the B29 lysine side chain.

11. The conjugate according to claim 10 wherein the B29 lysine is a monoconjugate.

12. The conjugate according to claim 10 wherein the B1 and B29 amino acids are both conjugated.

13. The conjugate according to claim 10 wherein the A1, B1 and B29 amino acids are all conjugated.

14. The conjugate according to claim 10 wherein the cell permeable peptide differs for each conjugate.

15. The peptide according to claim 1 wherein the cell-permeable peptide contains a peptide having SEQ ID No. 2 and at least one independent repeating unit of the 11 amino acid sequence of SEQ ID No: 1.

16. A pharmaceutical composition comprising a conjugate according to claim 1 and a pharmaceutically acceptable carrier or diluent.

17. A pharmaceutical composition comprising an effective amount of a conjugate according to claim 1, in admixture with one or more pharmaceutically acceptable carriers, diluents or excipients, for administration by a route selected from the group consisting of oral, intravenous, intramuscular, subcutaneous, intranasal, oral inhalation, intrarectal, intravaginal and intraperitoneal.

18. The pharmaceutical composition according to claim 17 for administration by a route selected from the group consisting of oral, intranasal, and oral inhalation.

19. A method for treating Type I or Type II diabetes in a subject in need thereof which comprises administering to said subject an effective amount of a conjugate according to claim 1.

20. The method according to claim 19 wherein the conjugate is administered by a route selected from the group consisting of oral, intravenous, intramuscular, subcutaneous, intranasal, oral inhalation, intrarectal, intravaginal and intraperitoneal means.

21. The method according to claim 20 wherein the conjugate is administered orally, intranasally or by oral inhalation.

22. A method for treating, including prophylaxis, of prediabetes and/or metabolic syndrome in a subject in need thereof which comprises administering to said subject an effective amount of a conjugate according to claim 1.

23. The method according to claim 22 wherein the conjugate is administered by a route selected from the group consisting of oral, intravenous, intramuscular, subcutaneous, intranasal, oral inhalation, intrarectal, intravaginal and intraperitoneal means.

24. The method according to claim 23 wherein the conjugate is administered orally, intranasally or by oral inhalation.

25. A pharmaceutical composition comprising a cell-permeable peptide of about 11 to about 50 amino acid residues comprising at least one residue of SEQ ID NO: 1 and a compound selected from the group consisting of an insulin compound, calcitonin, calcitonin gene related peptide, parathyroid hormone, and luteinizing hormone-releasing hormone, and a pharmaceutically acceptable carrier or diluent.

26. The peptide according to claim 25 wherein the cell-permeable peptide contains at least 2 repeating units of the 11 amino acid sequence of SEQ ID No: 1 or the 12 amino acid sequence of SEQ ID No.:2.

27. The peptide according to claim 25 wherein the cell-permeable peptide contains at least 3 repeating units of the 11 amino acid sequence of SEQ ID No: 1 or the 12 amino acid sequence of SEQ ID No.:2.

28. The peptide according to claim 25 wherein the cell-permeable peptide contains at least 4 repeating units of the 11 amino acid sequence of SEQ ID No: 1 or the 12 amino acid sequence of SEQ ID No.:2.

29. The peptide according to claim 25 wherein the repeating units of the 11 amino acid sequence of SEQ ID No: 1 or the 12 amino acid sequence of SEQ ID No.:2. are separated by at least one or more amino acid residues.

30. The peptide according to claim 29 wherein the separated amino acid residue is alanine.

31. The conjugate according to claim 25 wherein the insulin is mammalian insulin.

32. The conjugate according to claim 31 wherein the mammalian insulin is selected from the group consisting of human insulin, bovine insulin compound, and porcine insulin compound.

33. A pharmaceutical composition comprising an effective amount of a cell-permeable peptide of about 11 to about 50 residues comprising at least one consecutive residue of SEQ ID NO: 1 and a compound selected from the group consisting of an insulin compound, a calcitonin, a calcitonin gene related peptide, parathyroid hormone, or a luteinizing hormone-releasing hormone, in admixture with one or more pharmaceutically acceptable carriers, diluents or excipients, for administration by oral intravenous, intramuscular, subcutaneous, intranasal, oral inhalation, intrarectal, intravaginal and intraperitoneal means.

34. The pharmaceutical composition according to claim 33 for administration orally, intranasally, or by oral inhalation.

35. A method for treating Type I or Type II diabetes in a subject in need thereof which comprises administering to said subject an effective amount of a pharmaceutical composition according to claim 33.

36. The method according to claim 35 wherein the composition is administered by oral, intravenous, intramuscular, subcutaneous, intranasal, oral inhalation, intrarectal, intravaginal or intraperitoneal means.

37. The method according to claim 35 wherein the composition is administered orally, intranasally or by oral inhalation.
